(19) United States
(12) Patent Application Publication
(10) Pub. No.: US 2024/0369546 A1
(43) Pub. Date: Nov. 7, 2024

(54) RED BLOOD CELL-DERIVED MAGNETIC IMMUNO-PARTICLE AND USE THEREOF

(71) Applicant: BELEMENT INC., Ulsan (KR)

(72) Inventors: Joo Hun KANG, Ulsan (KR); Sung Jin PARK, Ulsan (KR); Jinmyoung JOO, Ulsan (KR)

(21) Appl. No.: 18/575,256

(22) PCT Filed: Oct. 27, 2023
(86) PCT No.: PCT/KR2023/016886

§ 371 Date: Jul. 24, 2024

(30) Foreign Application Priority Data
Oct. 28, 2022 (KR) 10-2022-0141702

(51) Int. Cl. G01N 33/543; A61M 1/36; G01N 33/555; G01N 33/569; G01N 33/68

(52) U.S. Cl. CPC G01N 33/54326; A61M 1/3618; A61M 1/362; G01N 33/555; G01N 33/56916; G01N 33/56938; G01N 33/56983; G01N 33/6896; G01N 2333/03; G01N 2333/135; G01N 2333/245; G01N 2333/31; G01N 2333/4709; G01N 2333/525; G01N 2333/535; G01N 2333/5406; G01N 2333/5412; G01N 2333/545; G01N 2800/26; G01N 2800/2821

(57) ABSTRACT

The present application relates to a erythrocyte-derived magnetic immune particle and uses thereof, according to an aspect, the erythrocyte-derived magnetic immune particle include an erythrocyte-derived cell membrane, which may minimize in vivo side effect, and may be used to detect and remove various type of substances (for example, a pathogenic substance, an inflammatory cytokine, blood glucose, a cancer-related substance, and a brain disease-related substance, etc.) from a sample with excellent efficiency, which may be useful for diagnosing, preventing, or treating various type of diseases, including an infectious disease, an inflammatory disease, diabetes, cancer, and brain disease.

### TECHNICAL FIELD

The present disclosure relates to an erythrocyte-derived magnetic immune particle and use thereof, which claims priority to Korean Patent Application No. 10-2022-0141702, filed on Oct. 28, 2022, with the Korean Intellectual Property Office, the disclosure of which is incorporated herein by reference.

### BACKGROUND ART

Worldwide, pathogens such as pathogenic microorganisms or antigens derived therefrom infect water systems and humans, causing many diseases.

Previously developed methods of detecting pathogens in blood have long detection times and low detection efficiency. For example, cell culture and polymerase chain reaction (PCR), which is a gene detection method, and the like, have been developed to detect microorganisms (Korean Public Patent No. 10-2018-0023545). Cell culture is a method of isolating pathogenic viruses from non-pathogenic viruses and identifying the same, and this method is determined by whether cytopathic effect (CPE) occurs. Generally, it takes a long time, about 1 weeks to about 4 weeks, for the cytopathic effect to be seen. Therefore, the cell culture method is less useful for determining the presence of harmful microorganisms and preparing countermeasures. In addition, a polymerase chain reaction method, which is one of the genetic diagnostic methods, is a method of amplifying a small amount of DNA or RNA, and its sensitivity, specificity, and rapidity are much better than those of the cell culture method, and accordingly, the disadvantages of the cell culture method can be overcome. However, if a trace amount of harmful microorganisms is present in the sample or a large amount thereof is lost during the nucleic acid extraction process, the result of the polymerase chain reaction method may be determined as being negative. In other words, it is possible that microorganisms are present even if they are not detected by polymerase chain reaction. In addition, the fundamental problem with the polymerase chain reaction method is that quantitative analysis is difficult and there is a high risk of false positives due to contamination. On the other hand, the treatment of diseases caused by infections still relies mostly on antibiotics administration methods. However, antibiotic administration has a critical disadvantage of causing blood cell depletion, hypersensitivity reactions, and toxic side effects to the nervous system, heart, kidneys, and liver. More recently, the emergence of ‘super bacteria’, which has resistance to antibiotics, has also lowered the success rate of the treatment of infectious diseases based on antibiotic administration. To solve these problems, there is a need to develop a method for detecting or removing pathogens that is safe due to no side effects when administered into the body while having high pathogen detection efficiency.

Furthermore, in addition to pathogenic substances such as a pathogen, inflammatory cytokine, blood glucose, cancer-related substance such as extracellular vesicle derived from a cancer cell or nucleic acid derived from a tumor cell, and a brain disease-related substance such as amyloid beta 42, amyloid beta 40, or tau protein, may be a marker for various diseases such as inflammatory disease, diabetes, cancer, and brain disease, or cause or worsen the various diseases. However, in the prior art, in order to detect these various disease-related substances present in the body, a method, a material, a device, a kit, etc. corresponding to each substance may be applied, resulting in high costs and reduced detection or analysis efficiency. Therefore, there is a need for a method that may diagnose, prevent, or treat various diseases by detecting or removing various disease-related substances present in the body in a single, simple method, and a method that may detect or remove disease-related substances with a broad spectrum and high efficiency, which has not been developed to date.

### DISCLOSURE

### Technical Problem

To solve the above problems, the inventors of the present disclosure have developed an erythrocyte-derived magnetic immune particle, wherein the erythrocyte-derived magnetic immune particle includes an erythrocyte-derived cell membrane, which may minimize in vivo side effects, and may detect various kinds of substances (for example, pathogenic substance, inflammatory cytokine, blood glucose, cancer-related substance, and brain disease-related substance, etc.) may be detected and removed from a sample with excellent efficiency, thereby confirming that the particle is useful for diagnosing, preventing, or treating various types of diseases, including an infectious disease, inflammatory disease, diabetes, cancer, and brain disease.

One objective of the present disclosure is to provide a magnetic immune particle including a cell membrane of erythrocyte origin; and a magnetic particle attached to the cell membrane.

Another objective of the present disclosure is to provide a composition for detecting or removing pathogenic substance, inflammatory cytokine, blood glucose, cancer-related substance, or brain disease-related substance including the magnetic immune particle.

Another objective of the present disclosure is to provide a composition for diagnosis of an infectious disease, inflammatory disease, diabetes, cancer, or brain disease including the magnetic immune particle.

Another objective of the present disclosure is to provide a method for detecting or removing pathogenic substance, inflammatory cytokine, blood glucose, cancer-related substance, or brain disease-related substance present in a sample from the sample, utilizing the magnetic immune particle.

Another objective of the present disclosure is to provide a method of providing information necessary for the diagnosis of an infectious disease, inflammatory disease, diabetes, cancer, or brain disease (or a method of diagnosing an infectious disease, inflammatory disease, diabetes, cancer, or brain disease) utilizing the magnetic immune particle.

Another objective of the present disclosure is to provide a method of preventing or treating an infectious disease, inflammatory disease, diabetes, cancer, or brain disease, utilizing the magnetic immune particle.

Another objective of the present disclosure is to provide a kit or device utilizing the magnetic immune particle.

Another objective of the present disclosure is to provide a method of producing the magnetic immune particle.

However, the technical challenge to be achieved by the present disclosure is not limited to those mentioned above, and other challenges not mentioned will be apparent to one of ordinary skill in the art from the following description.

### Technical Solution

Unless otherwise defined, all technical and scientific terms used herein have the same meaning as would be commonly understood by a person skilled in the art to which the present disclosure belongs. In general, the nomenclature used herein is well known and in common use in the art.

One aspect provides a magnetic immune particle including a cell membrane of erythrocyte origin; and a magnetic particle attached to the cell membrane.

The magnetic immune particle is a magnetic particle attached to an erythrocyte-derived cell membrane that may capture various substances, specifically, substance to be captured, such as a pathogenic substance, inflammatory cytokine, blood glucose, cancer-related substance, brain disease-related substance, and the like. Additionally, the magnetic immune particle that capture the substance to be captured may be isolated or removed from a sample such as blood by a magnetic field. Thus, the magnetic immune particle may be used to detect, isolate, or remove the substance to be captured from a sample, such as blood. As used herein, the term “magnetic immune particle” may be used interchangeably with “magnetic nanoparticle,” “magnetic nanoimmune particle,” “cell membrane-magnetic particle complex,” “erythrocyte-derived magnetic immune particle,” and the like.

The techniques proposed herein are characterized by 1) preparing a magnetic immune particle wherein the magnetic particle is attached to an erythrocyte-derived cell membrane, 2) contacting the magnetic immune particle with a sample to cause the magnetic immune particle to capture a target substance to be captured (for example, a pathogenic substance) present in the sample, and 3) isolating the magnetic immune particle capturing the substance to be captured from the sample by a magnetic field to detect the substance to be captured in the sample and/or remove the substance to be captured from the sample.

As used herein, the term “attachment” may refer to being located on the outside or inside of the cell membrane bilayer. For example, the term may refer to a form in which a magnetic particle is directly bonded to the outside or inside of the cell membrane bilayer, or a form in which the magnetic particle is absorbed and trapped (or embedded, or encapsulated) inside the cell membrane, but is not limited thereto.

The pathogenic substance may include one or more types selected from the group consisting of bacteria, fungi, virus, parasite, prion, and toxin, but includes without limitation any pathogenic substance that may be captured by a cell membrane. The pathogenic substance may be one that cause an infectious disease in the body, such as malaria, etc. Additionally, the pathogenic substance may include a cell infected with the pathogenic substance, for example a cell infected with a malaria larvae, etc. Additionally, the pathogenic substance may include a pathogen. The pathogenic substance may also include, but is not limited to, pathogenic substance present in a biological sample (for example, blood) isolated from a patient suffering from a disease (for example, infectious disease, inflammatory disease, diabetes, cancer, brain disease, etc.).

In an embodiment, the pathogenic bacteria may be any type of gram-positive bacteria or gram-negative bacteria. More specifically, the pathogenic bacteria may include, but is not limited to, one or more types selected from the group consisting of Enterobacteriaceae spp, Enterococcus spp, Citrobacter spp, Staphylococcus spp, Klebsiella spp, Pseudomonas spp, Acinetobacter spp, Salmonella spp, and Streptococcus spp, Escherichia spp, Mycobacterium spp, Mycoplasma spp, Vibrio spp, and Shigella spp, Campylobacter spp, Chlamydia spp, and the above bacteria that have acquired antibiotic resistance.

Additionally, in an embodiment, the pathogenic fungi may include one or more types selected from the group consisting of Candida spp, Aspergillus spp, Trichophyton spp, and Cladophialophora spp, but is not limited thereto.

Additionally, in an embodiment, the pathogenic virus may include, but is not limited to, one or more types selected from the group consisting of Adenoviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae. More specifically, the pathogenic virus may include, but is not limited to, one or more types selected from the group consisting of Respiratory syncytial virus (RSV), Zika virus (ZIKV), Human coronavirus 229E (HCov229E), Human coronavirus OC43 (HCoV-OC43), Cytomegalovirus (CMV), Severe acute respiratory syndrome coronavirus 1 (SARS-COV-1), Severe acute respiratory syndrome coronavirus 2 (SARS-COV-2), a variant of SARS-COV-2 (Alpha variant, Beta variant, Delta variant, etc.), Ebola virus, and Dengue virus.

Additionally, in an embodiment, the parasite may include, but is not limited to, a malaria larvae.

Additionally, in an embodiment, the prion may include an infectious protein pathogen that cause an infectious disease including, but not limited to, scrapie, bovine spongiform encephalopathy, Creutzfeldt-Jakob disease, etc.

Additionally, in an embodiment, the toxin may include any agent that has pathogenic properties, such as causing disease, and may include, but is not limited to, a pathogenic substance (for example, an antigenic protein) derived from a pathogenic bacterium, fungi, virus, or parasite. For example, the toxin may include, but is not limited to, endotoxin (Lipopolysaccharide, LPS), Zika virus (ZIKV) envelope protein, SARS-COV-2 spike protein, or SARS-COV-2 variant viral spike protein (Alpha variant spike protein, Beta variant spike protein, or Delta variant spike protein), etc.

The inflammatory cytokine may be included without limitation as long as it is a cytokine that causes or worsens inflammation, for example, the inflammatory cytokine may include one or more types selected from the group consisting of tumor necrosis factor-α (TNF-α), interleukin 4 (IL-4), interleukin 6 (IL-6), interleukin-1 beta (IL-1β), interleukin-1 alpha (IL-1α), interleukin 8 (IL-8), interferon gamma (IFN-γ), and granulocyte-macrophage colony-stimulating factor (GM-CSF), but is not limited thereto. The inflammatory cytokine may also include, but are not limited to, inflammatory cytokine present in a biological sample (for example, blood) isolated from a patient suffering from a disease (for example, infectious disease, inflammatory disease, diabetes, cancer, brain disease, etc.).

The blood glucose may include, but is not limited to, glucose present in a biological sample (for example, blood) isolated from a patient suffering from a disease (for example, infectious disease, inflammatory disease, diabetes, cancer, brain disease, etc.).

The cancer-related substance may include a cancer (tumor) cell (for example, a circulating tumor cell (CTC), blood cancer (Leukemia, Myeloma, etc.)), a cancer causing substance, a cancer metastasis causing substance, a cancer cell-derived substance, cancer cell metabolite, metabolite of cancer disease development or progression, and the like, and for example, may include a cancer cell-derived extracellular vesicle, cancer cell (for example, circulating tumor cell) derived nucleic acid, or a combination thereof, but is not limited thereto. The cancer-related substance may also include, but is not limited to, cancer-related substance present in a biological sample (for example, blood) isolated from a patient suffering from a disease (for example, infectious disease, inflammatory disease, diabetes, cancer, brain disease, etc.).

The brain disease-related substance may include a brain disease-induced substance, metabolite of brain disease development or progression, etc., for example, may include, an amyloid beta (Aβ) protein (for example, amyloid beta 42 (Aβ42), amyloid beta 40 (Aβ40), etc.), tau protein, or a combination thereof, but is not limited thereto. The amyloid beta protein may include, but is not limited to, amyloid beta monomer, amyloid beta dimer, amyloid beta oligomer, amyloid beta plaque, and the like. The brain disease-related substance may also include, but is not limited to, brain disease-related substance present in a biological sample (for example, blood) isolated from a patient suffering from a disease (for example, infectious disease, inflammatory disease, diabetes, cancer, brain disease, etc.).

As used herein, the term “capture” refers to the binding or connection of a substance to be captured with the magnetic immune particle. Specifically, the term “capture” may refer to the substance to be captured is bound or connected to the surface or interior of the cell membrane of the magnetic immune particle. As used herein, the term “capture” may be used interchangeably with the term “capture,” “adsorption,” “absorption,” “binding,” “connection,” and the like.

The erythrocyte or erythrocyte-derived cell membrane may be derived from a cell of one or more types of subject selected from the group consisting of, but not limited to, a human, primate such as a monkey, etc., rodent such as a rat and mice, etc., bovines such as a horse, cattle, pig, sheep, and goat, etc., mammal such as a horse, dog, and cat, etc., a bird, fish, reptile, amphibian, crustacean, and insect. The erythrocyte-derived cell membrane has excellent safety for in vivo administration due to not causing immune rejection when administered to the body.

As used herein, the term “cell membrane” refers to the cell membrane itself present in the cell, or a cell membrane isolated from the cell by a typical method, for example sonication, use of osmotic pressure difference, extrusion, or the like (specifically, an intact cell membrane, a portion or fragment of a cell membrane, a membrane formed by reassembling portions or fragments of a cell membrane, or the like).

The erythrocyte-derived cell membrane may include, express, or overexpress one or more types selected from the group consisting of a complement receptor (CR), cluster of differentiation (CD) molecule, glycophorin, duffy antigen receptor for chemokines (DARC), glucose transporter (for example, glucose transporter 1 (GLUT1), etc.), and monocarboxylate transporter (MCT), but is not limited thereto. By expressing one or more types selected from the group consisting of a complement receptor, a cluster of differentiation molecule, a glycophorin, a duffy antigen receptor for chemokines, a glucose transporter, and a monocarboxylate transporter, the cell membrane may capture or uptake (endocytosis) the magnetic particle or the substance to be captured, or bind or attach to the magnetic particle or the substance to be captured.

The erythrocyte-derived cell membrane may include, express, or overexpress a complement receptor, a cluster of differentiation molecule, a glycophorin, a duffy antigen receptor for chemokines, a glucose transporter, a monocarboxylate transporter, or a combination thereof (for example, complement receptor 1 (CR1), glycophorin A (GYPA), or a combination thereof). Overexpression may refer to increased expression of a complement receptor, a cluster of differentiation molecule, a glycophorin, a duffy antigen receptor for chemokines, a glucose transporter, a monocarboxylate transporter, or a combination thereof (for example, CR1, GYPA, or a combination thereof), compared to the cell membrane of a normal erythrocyte.

Erythrocytes that have a cell membrane overexpressing a complement receptor, a cluster of differentiation molecule, a glycophorin, a duffy antigen receptor for chemokines, a glucose transporter, a monocarboxylate transporter, or a combination thereof (for example, CR1, GYPA, or a combination thereof) may be prepared according to methods known in the art. For example, a method of performing transformation by genetic recombination to increase the expression of a complement receptor, a cluster of differentiation molecule, a glycophorin, a duffy antigen receptor for chemokines, a glucose transporter, a monocarboxylate transporter, or a combination thereof (for example, CR1, GYPA, or a combination thereof) in erythrocyte precursors using genetic engineering and then differentiating the transformed erythrocyte precursors into erythrocyte may be used. Specifically, a method of increasing the copy number of a gene in an erythroid precursor cell by transfection of a foreign gene encoding a complement receptor, a cluster of differentiation molecule, a glycophorin, a duffy antigen receptor for chemokines, a glucose transporter, a monocarboxylate transporter, or a combination thereof (for example, CR1, GYPA, or a combination thereof) into the erythroid precursor cell may be utilized, and the method may be any method known in the art, for example, but not limited to, introducing into the erythroid precursor cell a vector operably linked to a gene encoding a complement receptor, a cluster of differentiation molecule, a glycophorin, a duffy antigen receptor for chemokines, a glucose transporter, a monocarboxylate transporter, or a combination thereof (for example, CR1, GYPA, or a combination thereof) that is host-independent or capable of replicating and functioning within the erythroid precursor cell.

Through the above-mentioned methods, etc., erythrocytes having a cell membrane overexpressing a complement receptor, a cluster of differentiation molecule, a glycophorin, a duffy antigen receptor for chemokines, a glucose transporter, a monocarboxylate transporter, or a combination thereof (for example, CR1, GYPA, or a combination thereof) may be prepared, and according to a method according to an embodiment of the present disclosure, the cell membrane is isolated from the erythrocytes, attaching a magnetic particle to the isolated cell membrane, thereby producing a magnetic immune particle including a erythrocyte-derived cell membrane overexpressing a complement receptor, a cluster of differentiation molecule, a glycophorin, a duffy antigen receptor for chemokines, a glucose transporter, a monocarboxylate transporter, or a combination thereof (for example, CR1, GYPA, or a combination thereof).

According to an embodiment, it was confirmed that for the magnetic immune particle including the erythrocyte-derived cell membrane, the surface molecules of the cell membrane, CR1, GYPA, or a combination thereof, contribute significantly to the capture ability (detection or removal ability) of the magnetic immune particle for a substance to be captured. Therefore, in the case of the magnetic immune particle including the erythrocyte-derived cell membrane overexpressing CR1, GYPA, or a combination thereof, the capture ability (detection or removal ability) for the substance to be captured may be significantly increased.

As used herein, the term “magnetic particle” refers to a particle that is responsive to a magnetic field and may be readily taken up by a cell, or may bind, attach, enter, entrap, enclose, or be trapped outside or inside a cell membrane. Specifically, the magnetic particle may include, but not limited to, one or more magnetic elements selected from the group consisting of, iron (Fe), nickel (Ni), cobalt (Co), manganese (Mn), bismuth (Bi), zinc (Zn), strontium (Sr), lanthanum (La), and cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), ruthenium (Lu), copper (Cu), silver (Ag), gold (Au), cadmium (Cd), mercury (Hg), aluminum (Al), gallium (Ga), indium (In), thallium (TI), calcium (Ca), barium (Ba), radium (Ra), platinum (Pt), and lead (Pd).

The magnetic element may be oxidized or surface modified. Specifically, iron may be oxidized and included in the magnetic immune particle in the form of iron oxide. The surface modification may be, but is not limited to, a surface modification by a metal, a surface modification by a functional group such as a carboxylic or amine group, a surface modification by a protein such as an antibody, streptavidin, or avidin, a surface modification by a carbohydrate, a surface modification by a polymer, or a surface modification by a lipid. The magnetic particle may be stabilized by the above modification.

The magnetic particle may be prepared and used by known methods, or may be purchased and used commercially.

The magnetic particle may be used as is or dispersed or suspended in an appropriate solvent (for example, buffer (PBS, saline, Tris-buffered saline, etc.), but not limited thereto.

The magnetic particle may have a small particle size such that each particle has a single magnetic zone. As a result, the magnetic particle may exhibit superparamagnetism, which is the property of being magnetic only in the presence of an external magnetic field. By creating the magnetic immune particle from the magnetic particle that exhibit superparamagnetism, the magnetic immune particle may be isolated simply and easily by applying an external magnetic field. Isolation by applying a magnetic field is not affected by the surrounding environment such as pH, temperature, ions, etc., and thus has excellent stability and sensitivity.

The magnetic particle may be selected from among all particles that are magnetic and have a particle size that may be attached to, introduced into, embedded in, or encapsulated in a cell membrane capable of capturing the substance to be captured. For example, the magnetic particle may be a magnetic particle having an average particle diameter of about 100 nm to about 500 nm or about 100 nm to about 300 nm, but are not limited thereto.

The magnetic particle may be included in a solution. The magnetic particle may be attached to the cell membrane while included in the solution. In an embodiment, the solution may include a medium, buffer, or combination thereof used to culture or differentiate cells, and may be the same as the medium for the magnetic immune particle.

The magnetic immune particle may include an outer surface including a cell membrane, and an inner core including a magnetic particle.

In the magnetic immune particle, the inner core may include one or more, for example, at least 1 but not more than 1,000,000 magnetic particles. Specifically, the inner core may include at least 1 but not more than 100,000, at least 1 but not more than 10,000, at least 1 but not more than 1,000, at least 1 but not more than 100, or at least 1 but not more than 10 magnetic particles. The number of magnetic particles included in the inner core may be appropriately formed according to the size of the magnetic particle, or the size of the magnetic immune particle. When the inner core includes two or more magnetic particles, the effect of capturing, detecting, or removing a substance to be captured (for example, a pathogenic substance) may be improved.

The average diameter of the magnetic immune particle is about 1 nm to about 30,000 nm, about 10 nm to about 30,000 nm, about 50 nm to about 30,000 nm, about 100 nm to about 30,000 nm, about 200 nm to about 30,000 nm, about 300 nm to about 30,000 nm, about 400 nm to about 30,000 nm, about 500 nm to about 30,000 nm, about 1 nm to about 20,000 nm, about 10 nm to about 20,000 nm, about 50 nm to about 20,000 nm, about 100 nm to about 20,000 nm, about 200 nm to about 20,000 nm, about 300 nm to about 20,000 nm, about 400 nm to about 20,000 nm, about 500 nm to about 20,000 nm, about 1 nm to about 10,000 nm, about 10 nm to about 10,000 nm, about 50 nm to about 10,000 nm, about 100 nm to about 10,000 nm, about 200 nm to about 10,000 nm, about 300 nm to about 10,000 nm, about 400 nm to about 10,000 nm, about 500 nm to about 10,000 nm, about 1 nm to about 5,000 nm, about 10 nm to about 5,000 nm, about 50 nm to about 5,000 nm, about 100 nm to about 5,000 nm, about 200 nm to about 5,000 nm, about 300 nm to about 5,000 nm, about 400 nm to about 5,000 nm, about 500 nm to about 5,000 nm, 1 nm to about 1,000 nm, about 10 nm to about 1,000 nm, about 50 nm to about 1,000 nm, about 100 nm to about 1,000 nm, about 200 nm to about 1,000 nm, about 300 nm to about 1,000 nm, about 400 nm to about 1,000 nm, about 500 nm to about 1,000 nm, about 1 nm to about 500 nm, about 10 nm to about 500 nm, about 50 nm to about 500 nm, about 100 nm to about 500 nm, about 200 nm to about 500 nm, about 300 nm to about 500 nm, about 400 nm to about 500 nm, about 150 nm to about 350 nm, or about 200 nm to about 300 nm, but is not limited thereto.

In the magnetic immune particle, the cell membrane may have the form of a vesicle. As used herein, the term “vesicle” may refer to a particle formed by extracting (isolating) a cell membrane from a cell by a known technique, such as sonication, using an osmotic pressure difference, or extrusion, followed by self-assembly of the isolated cell membrane, or reassembly by extrusion.

An example of a manufacturing process of a magnetic immune particle according to an embodiment is schematically shown in FIG. 1. Specifically, according to the Type 1 method of FIG. 1, erythrocytes extracted from a subject may be administered to a solution (for example, blood, aqueous solution, purified water, buffer, medium, etc.) including a magnetic particle to cause the magnetic particle to be absorbed (or included, incorporated) into the cells by cellular uptake, thereby generating erythrocytes (for example, magnetic immune cells) or a erythrocyte analog internally containing the magnetic particle. The erythrocyte (for example, magnetic immune cell) containing the magnetic particle therein may perform the original function of an erythrocyte and at the same time exhibit magnetism or be influenced by magnetic fields due to the magnetic particle contained therein.

Furthermore, according to the Type 2 method of FIG. 1, a magnetic immune particle may be formed using a cell membrane isolated (or purified) from an erythrocyte. Specifically, by isolating (or purifying) the cell membrane from the erythrocyte by a typical method (for example, using osmotic pressure difference, sonication, extrusion, etc.), and by extruding or sonicating the erythrocyte-derived cell membrane and the magnetic particle obtained therefrom to incorporate the magnetic particle inside the erythrocyte-derived cell membrane, a magnetic immune particle containing the magnetic particle inside erythrocyte-derived cell membrane may be generated. More specifically, in order to isolate (or purify) the cell membrane from the erythrocyte, the erythrocyte may be administered to a hypo-osmotic solution to form pores in the cell membrane that allow the hypo-osmotic solution to move into the erythrocyte and cause the erythrocyte to swell, while simultaneously allowing the erythrocyte intracellular organelles to escape into the extracellular environment. Through the pores, the escaped intracellular organelles may be separately removed by centrifugation, the cell-derived membrane alone may be isolated (or purified), and the isolated (or purified) erythrocyte-derived cell membrane may be sonicated to break the cell membrane into smaller pieces. The erythrocyte-derived cell membrane and magnetic particle may be mixed and subjected to extrusion or sonication to generate a magnetic immune particle containing the magnetic particle inside the cell membrane.

Additionally, as shown in FIG. 1, according to a Type 3 method, a cell membrane isolated (or purified) from the erythrocyte may be utilized to form the magnetic immune particle. Specifically, by isolating (or purifying) the cell membrane from the erythrocyte by a typical method (for example, using osmotic pressure difference, extrusion, etc.), and by mixing then sonicating the erythrocyte-derived cell membrane and the magnetic particle obtained therefrom to incorporate the magnetic particle into the erythrocyte-derived cell membrane, a magnetic immune particle containing the magnetic particle inside erythrocyte-derived cell membrane may be generated.

The magnetic particle included in the magnetic immune particle may be a monodispersed magnetic particle.

According to an embodiment, the magnetic immune particle may be obtained by extruding and/or sonicating a mixture of the magnetic particle (for example, monodisperse magnetic particle) with erythrocytes or the cell membrane isolated from erythrocytes.

The monodisperse magnetic particle may refer to a plurality of magnetic particles having a high degree of uniformity (or uniformity) in particle size, such as a polydispersity index (PDI) of about 0.17 or less. The polydispersity index may be defined as the square of the standard deviation of the particle size (for example, diameter) divided by the average of the particle size (for example, diameter), and may refer to a value between 0 and 1, with a value closer to 0 (for example, about 0.17 or less) indicating that the particle are monodisperse particle with a high degree of uniformity in size. Additionally, the monodisperse magnetic particle may have a diameter of about 200 nm to about 300 nm. The monodisperse magnetic particle may be prepared by a general method known in the art, and magnetic particle(s) having a uniform size (or a high degree of uniformity) (for example, having a diameter distribution of about 200 to about 300 nm and a polydispersity index of about 0.17 or less) that are not limited by the method of preparation may be used as the monodisperse magnetic particle without limitation.

Specifically, the magnetic particle may be a magnetic particle(s) (for example, monodisperse magnetic particle(s)) having a polydispersity index of about 0.17 or less, about 0.1 or less, about 0.05 or less, about 0.03 or less, about 0.02 or less, more than about 0 to 0.17, more than about 0 to 0.1, more than about 0 to 0.05, more than about 0 to 0.03, more than about 0 to 0.02, about 0.01 to 0.03, or about 0.02.

According to an embodiment, in the case of the magnetic immune particle obtained by extruding and/or sonicating a mixture of the monodisperse magnetic particle(s) with erythrocytes or the cell membrane isolated from erythrocytes, the capture ability (detection ability or removal ability) for the pathogenic substance, inflammatory cytokine, blood glucose, cancer-related substance, or brain disease-related substance may be further increased compared to the magnetic immune particle prepared using a polydisperse magnetic particle (for example, polydisperse magnetic particle(s) of inhomogeneous size with a polydispersity index of greater than about 0.17).

The magnetic immune particle may function similarly to an erythrocyte, as the outer membrane include the cell membrane component (lipid bilayer, membrane protein, receptor, etc.) of the erythrocyte. Furthermore, the magnetic immune particle may be magnetic or affected by a magnetic field due to magnetic particle contained therein.

Another aspect provides a composition for detecting or removing a substance to be captured, including the magnetic immune particle. The substance to be captured may include one or more types selected from the group consisting of a pathogenic substance, an inflammatory cytokine, blood glucose, a cancer-related substance, and a brain disease-related substance. The pathogenic substance, inflammatory cytokine, blood glucose, cancer-related substance, and brain disease-related substance are as described above.

Another aspect provides a composition for diagnosing an infectious disease, inflammatory disease, diabetes, cancer, or brain disease including the magnetic immune particle.

As used herein, the term “detect” may include determining the presence of a substance to be captured in a sample, or detecting the substance to be captured present in a sample.

As used herein, the term “remove” may refer to removing a substance to be captured present in a sample by isolating the substance to be captured from the sample.

The magnetic immune particle may use an erythrocyte-derived cell membrane that may capture the substance to be captured, and may bind to the substance to be captured or capture the substance to be captured inside the cell membrane depending on the characteristics of the erythrocyte from which the cell membrane is derived. Additionally, the magnetic immune particles that capture the substance to be captured may be collected or concentrated in a magnetic field area by applying a magnetic field, and thereby isolated from the sample. Based on this principle, a composition including the magnetic immune particle may be used to detect the captured substance to be captured in a sample, remove the substance to be captured from a sample, or diagnose or treat a disease in a subject.

In the case of a magnetic immune particle utilizing a typical targeting substance (for example: an antibody), binding to a pathogenic substance was achieved by forming a targeting substance capable of binding to the pathogenic substance on the surface or inside the magnetic particle. Therefore, it is not possible to effectively target a pathogenic substance without accurate information on the specific antigen of the pathogenic substance to be targeted, and there is great difficulties in targeting multiple types of pathogenic substances at the same time. In addition, since antibodies had to be used, there was a problem that synthesis was very difficult and costly.

However, since the magnetic immune particle may use erythrocytes isolated from living organisms or cell membranes derived therefrom, the magnetic immune particle has the advantage of being able to capture various types of unknown substance to be captured (for example, a pathogenic substance, an inflammatory cytokine, blood glucose, a cancer-related substance, a brain disease-related substance, and the like) at once by utilizing the system and characteristics of the erythrocytes themselves, and thus may be useful for the detection or removal of various substances.

The composition may rapidly detect or remove contaminants (for example, bacteria, fungi, virus, other microorganism, toxin (for example, endotoxin, etc.), contaminating compound, etc.) present in trace amounts in various foods including drinking water and beverage, sanitary product, environmental sample, etc., thus may be used for safety evaluation of food, sanitary product, environmental sample, etc. Additionally, the composition may detect or remove contaminant present in a biological sample.

The diagnostic compositions may be used to diagnose an infectious disease, inflammatory disease, diabetes, cancer, or brain disease in a subject by detecting substances (for example, a pathogenic substance, an inflammatory cytokine, blood glucose, a cancer-related substance, a brain disease-related substance, and the like) captured in the magnetic immune particle.

As used herein, the term “diagnosing a disease” may include determining whether a subject currently or previously has a particular disease, or determining whether a subject is infected with a substance capable of causing a particular disease.

The infectious disease may include, but is not limited to, one or more types selected from the group consisting of systemic or localized infection (for example, pathogenic bacterial or viral infection), inflammation, sepsis, and poisoning by toxins. Additionally, any disease caused by infection with the above-described substance to be captured (for example, pathogenic substance) is included without limitation. Specifically, the infectious disease may include, but is not limited to, one or more types selected from the group consisting of malaria, Mycobacterium tuberculosis, pneumonia, food poisoning, tetanus, typhoid, diphtheria, syphilis, Hansen's disease, chlamydial infection, smallpox, influenza, mumps, measles, chickenpox, ebola, rubella, coronavirus infection, scrapie, bovine spongiform encephalopathy, bacteremia, and Creutzfeldt-Jakob disease. The infectious disease may also include, but is not limited to, an infectious disease that a patient with another disease (for example, inflammatory disease, diabetes, cancer, brain disease, etc.) has or may additionally have.

The above inflammatory diseases may include, but are not limited to, one or more type selected from the group consisting of a systemic inflammatory response, cytokine release syndrome (for example, cytokine storm), atopic dermatitis, encephalitis, inflammatory enteritis, chronic obstructive pulmonary disease, pulmonary hemolytic shock, pulmonary fibrosis, undifferentiated spondyloarthropathy, undifferentiated arthropathy, and arthritis, inflammatory osteolysis, (chronic) inflammatory diseases caused by viral or bacterial infections, colitis, inflammatory bowel disease, type 1 diabetes, rheumatoid arthritis, reactive arthritis, osteoarthritis, psoriasis, vaccinia, osteoporosis, atherosclerosis, myocarditis, endocarditis, pericarditis, cystic fibrosis, hashimoto's thyroiditis, graves' disease, leprosy, syphilis, lyme, borreliosis, neurogenic-borreliosis, tuberculosis, sarcoidosis, lupus, discoid lupus, chilblains, lupus nephritis, systemic lupus erythematosus, macular degeneration, uveitis, irritable bowel syndrome, crohn's disease, sjogren's syndrome, fibromyalgia, chronic fatigue syndrome, chronic fatigue immune deficiency syndrome, myalgic encephalomyelitis, amyotrophic lateral sclerosis, parkinson's disease, and multiple sclerosis. The inflammatory disease may also include, but is not limited to, an inflammatory disease that a patient with another disease (for example, infectious disease, diabetes, cancer, brain disease, etc.) has or may additionally have.

The diabetes may also include, but is not limited to, a diabetes that a patient with another disease (for example, infectious disease, inflammatory disease, cancer, brain disease, etc.) has or may additionally have.

The cancer refers to any disease caused by cells that have an aggressive characteristic, in which the cells divide and grow beyond normal growth limits, an invasive characteristic, in which the cells infiltrate surrounding tissue, or a metastatic characteristic, in which the cells spread to other parts of the body. The cancer may include both solid cancer and non-solid cancer, and may include, for example, but are not limited to, one or more types selected from the group consisting of hematologic cancer, breast cancer, ovarian cancer, cervical cancer, colon cancer, lung cancer, liver cancer, brain cancer, esophageal cancer, prostate cancer, pancreatic cancer, thyroid cancer, colorectal cancer, and renal cancer. The cancer may also include, but is not limited to, a cancer that a patient with another disease (for example, infectious disease, inflammatory disease, diabetes, brain disease, etc.) has or may additionally have.

The brain disease may be, for example, but not limited to, a degenerative brain disease. Specifically, the brain disease may include, but is not limited to, Alzheimer's disease or Parkinson's disease etc. The brain disease may also include, but is not limited to, a brain disease that a patient with another disease (for example, infectious disease, inflammatory disease, diabetes, cancer, etc.) has or may additionally have.

The composition may further include an opsonin. The opsonin may include, but is not limited to, one or more types selected from the group consisting of MBL (Mannose binding lectin), FCN-1 (Ficolin-1), FCN-2 (Ficolin-2), FCN-3 (Ficolin-3), CL-10 (collectin-10), CL-11 (collectin-11), C3b, C1q (complement component 1q), Immunoglobulins, antibodies, C4b, surfactant protein A, surfactant protein D, pentraxins, and C-reactive protein.

According to an embodiment, the magnetic immune particle included in the composition may be further enhanced in the capture ability (detection or removal ability) of the substance to be captured (for example, a pathogenic substance) by the opsonin. In particular, compared to other opsonins, a specific opsonin, such as MBL, FCN-1, FCN-2, FCN-3, CL-10, CL-11, may further enhance the capture ability (detection or removal ability) of the magnetic immune particle to a substance to be captured (for example, a pathogenic substance).

The compositions may further include, but are not limited to, a solvent, a diluent, a binder, a lubricant, a disintegrant, a buffer, a suspension agent, an isotonic agent, a viscosity modifier, a vehicle, a dispersant, a lubricant, an excipient, a stabilizer, a pH adjuster, a preservative, and the like.

Among the terms or elements mentioned in the composition, those mentioned in the description of the magnetic immune particles are understood to be the same as previously mentioned in the description of the magnetic immune particles.

Another aspect provides a method of detecting or removing a substance to be captured present in a sample from the sample, including contacting the magnetic immune particle with a sample and mixing them; and applying a magnetic field to the mixed sample. The substance to be captured may include one or more types selected from the group consisting of a pathogenic substance, an inflammatory cytokine, blood glucose, a cancer-related substance, and a brain disease-related substance. The pathogenic substance, inflammatory cytokine, blood glucose, cancer-related substance, and brain disease-related substance are as described above.

Another aspect provides a method of providing information necessary to diagnose a disease (or a method of diagnosing a disease), including contacting and mixing the magnetic immune particle with a sample isolated from a subject; and applying a magnetic field to the mixed sample.

Another aspect provides a method of preventing or treating a disease in a subject, including contacting and mixing the magnetic immune particle with a sample (for example, blood) isolated from the subject; applying a magnetic field to the mixed sample to remove the magnetic immune particle from the sample; and injecting the magnetic immune particle-removed sample back into the subject.

The disease may include an infectious disease, an inflammatory disease, diabetes, cancer, or a brain disease. Wherein the infectious disease, inflammatory disease, diabetes, cancer, or brain disease is as described above.

In the method above, the sample may be one or more types selected from the group consisting of a biological sample (for example, blood (for example, whole blood), plasma, serum, lymphatic fluid, cerebrospinal fluid, or other body fluids, or cells, or tissues) isolated from an animal (including or not including humans), drinking water (for example, groundwater, tap water, bottled water, purified water, medicinal water, or the like), various foods, various sanitary products, tableware, kitchenware, and environmental samples (for example, soil, seawater, stream water, etc.) acting directly on a living organism, etc., but is not limited thereto, and may be any other object requiring detection and/or removal of the substance to be captured. The sample may itself be a fluid, or may be in the form of a suspension suspended in an appropriate medium (for example, purified water, sterile buffer, etc.).

The contacting and mixing the magnetic immune particle with the sample, which may be performed in vitro, may include culturing the magnetic immune particle with the sample in vitro. In this phase, the magnetic immune particle may bind (in other words, capture) to the substance to be captured present in the sample.

The culturing in vitro may be performed under a typical condition using a medium, buffer, saline, drinking water, the biological sample itself, or the like that are typically used for cell culture. For example, the phase may include culturing at a temperature of about 0° C. to 40° C. or about 2° C. to 38° C. for about 1 second to 96 hours, about 1 second to 48 hours, about 1 second to 24 hours, for example, about 1 second to 12 hours, about 1 second to 6 hours, about 1 second to 120 minutes, or about 1 second to 60 minutes.

Further, the applying the magnetic field may be performed in vitro.

The method may additionally include, after the applying a magnetic field to the mixed sample, isolating (or removing) magnetic immune particle from the sample using the applied magnetic field (magnetic force). The isolated magnetic immune particle may include a magnetic immune particle that have bound to the substance to be captured in the sample, as well as a magnetic immune particle that have failed to bind to any substance. This phase may be performed in vitro, whereby the substance to be captured and magnetic immune particle are removed from the sample.

A method of providing information necessary for the diagnosis of the disease (or a method of diagnosing a disease) may further include the detecting and/or analyzing the substance bound or captured by the magnetic immune particle. In addition, the method of providing information necessary for the diagnosis of the disease (or method of diagnosing a disease) may further include the determining or predicting the type of disease of the subject providing the sample, or determining information for the determination or prediction, from information obtained in the detecting and/or analyzing phase. For example, if the substance bound or captured by the magnetic immune particle is analyzed to be the pathogenic substance, and the amount is analyzed to be above a certain threshold, it may be determined or predicted that the subject providing the sample has, or is likely to have, the infectious disease. In addition, if, for example, the substance bound or captured by the magnetic immune particle is analyzed to be the inflammatory cytokine, and the amount is analyzed to be above a certain threshold, it may be determined or predicted that the subject providing the sample has, or is likely to have, the inflammatory disease. Additionally, for example, if the substance bound or captured by the magnetic immune particle is analyzed to be the blood glucose, and the amount is analyzed to be above a certain threshold, it may be determined or predicted that the subject providing the sample has or is likely to have, the diabetes. Additionally, if, for example, the substance bound or captured by the magnetic immune particle is analyzed to be the cancer-related substance, and the amount is analyzed to be above a certain threshold, it may be determined or predicted that the subject providing the sample has, or is likely to have, the cancer. Furthermore, if, for example, the substance bound or captured by the magnetic immune particle is analyzed to be the brain disease-related substance, and the amount is analyzed to be above a certain threshold, it may be determined or predicted that the subject providing the sample has, or is likely to have, the brain disease.

The prevention or treatment method may additionally include the applying a magnetic field to the mixed sample or the isolating (or removing) the magnetic immune particle from the sample using an applied magnetic field (magnetic force), followed by the injecting the sample from which the magnetic immune particle have been removed back into the body of the subject who provided the sample in vitro. The magnetic immune particle removed from the sample may include a magnetic immune particle that have bound to the substance to be captured in the sample, as well as a magnetic immune particle that have failed to bind to any substance. Accordingly, the sample injected back into the body of the subject may have both the substance to be captured and magnetic immune particle removed.

The method may additionally include analyzing the substance captured in the magnetic immune particle isolated (or removed) from the sample. The analysis may be performed by a mean, method, or device commonly used for the analysis of the substance to be captured.

In the above method, in order to facilitate measurement of the magnetic immune particle, the magnetic immune particle may be obtained using cells (cell membranes) and/or a magnetic particle labeled with a detectable labeling substance. The labeling substance may be any substance (small molecule compound or protein or poly/oligopeptide or the like) detectable by a typical method, and may be one or more types selected from the group consisting of, for example, a fluorescent substance, a luminescent substance and the like.

The method may further include the introducing or injecting one or more types selected from the group consisting of opsonin, blood, and plasma prior to applying the magnetic field. The opsonin may include, but is not limited to, one or more types selected from the group consisting of MBL (Mannose binding lectin), FCN-1 (Ficolin-1), FCN-2 (Ficolin-2), FCN-3 (Ficolin-3), CL-10 (collectin-10), CL-11 (collectin-11), C3b, C1q (complement component 1q), Immunoglobulins, antibodies, C4b, surfactant protein A, surfactant protein D, pentraxins, and C-reactive protein. According to an embodiment, in the above method, the capture ability (detection or removal ability) for a substance to be captured (for example, a pathogenic substance) may be further enhanced by introducing or injecting the opsonin. In particular, compared to other opsonins, a specific opsonin, such as MBL, FCN-1, FCN-2, FCN-3, CL-10, CL-11, may further enhance the capture ability (detection or removal ability) of the magnetic immune particle to a substance to be captured (for example, a pathogenic substance).

The blood or plasma may be blood or plasma isolated from the subject providing the sample or from another subject (for example, another person). For example, the blood or plasma may be isolated from the subject who provided the sample before or after the subject contracted the disease, or isolated from another subject (for example, another person) who was infected with the particular pathogenic substance, or isolated from another subject (for example, another person) who was vaccinated against the particular pathogenic substance. The blood or plasma may be enriched with an antibody or an opsonin to further enhance the capture ability (detection or removal ability) of the magnetic immune particle to a substance to be captured (for example, a pathogenic substance).

In the above method, the introducing or injecting one or more types selected from the group consisting of opsonin, blood, and plasma may be performed at any phase prior to applying the magnetic field. For example, prior to the contacting and mixing the magnetic immune particle with the sample, the magnetic immune particle or sample may be mixed with the one or more types selected from the group consisting of an opsonin, blood, and plasma, or during or after the contacting and mixing the magnetic immune particle with the sample, the one or more types selected from the group consisting of the opsonin, blood, and plasma may be further added.

The subject may be a human or a non-human animal (for example, a mammal, etc.). The method may be performed in vitro by applying a magnetic immune particle-based hemodialysis or magnetic immune particle-based extracorporeal circulation method.

As used herein, the term “prevention” refers to any act that prevent the occurrence of a disease, inhibit the disease, or delay progression of the disease.

The term “treatment” as used herein refers to both therapeutic treatment and preventive or prophylactic measure. In addition, the term also refers to any action that improve, alleviate, or beneficially alter the symptoms of a disease.

The method may be implemented in the form of a kit including the magnetic immune particle or a device utilizing the magnetic immune particle. The kit or device may be, but is not limited to, a magnetic immune particle-based hemodialysis kit or device, or a magnetic immune particle-based extracorporeal circulation kit or device. In addition, the kit or device may be implemented in the form of a fluidic device (for example, a microfluidic device), or may include a fluidic device (for example, a microfluidic device).

According to an embodiment, the method may be performed by a hemodialysis kit or device, or an extracorporeal circulation kit or device.

Schematic diagrams of the kit or device (for example, a hemodialysis kit or device, or an extracorporeal circulation kit or device) are illustrated in FIG. 6 and FIG. 9, but are not limited thereto.

Specifically, the kit or device (for example, hemodialysis kit or device, or extracorporeal circulation kit or device) may include a reaction unit and a magnetic field forming unit.

The reaction unit may refer to a portion where the magnetic immune particles and a sample are brought into contact and mixed, cultured, or reacted, or a reactant that causes a reaction by contact between the magnetic immune particle and the sample is introduced. In the kit or device, the magnetic immune particle may be included in the reaction unit, or may be applied to the reaction unit in the form of a reactant that has been previously reacted with the sample, or may be provided separately from the reaction unit, and may be provided in the form of a dispersion dispersed in an appropriate medium (for example, a buffer). In the reaction unit, the magnetic immune particle may bind (in other words, capture) to a substance to be captured present in the sample.

The magnetic field forming unit may refer to a portion that form a magnetic field. For example, the magnetic field forming unit may include one or more means for applying a magnetic field, such as a magnet (for example, an electromagnet by electromagnetic induction, a permanent magnet, or the like). The magnetic field forming unit may be included in the reaction unit, may be provided separately from the reaction unit, or may be provided integrally with all or part of the reaction unit. If the magnetic field forming unit exists separately from the reaction unit, the reaction unit and the magnetic field forming unit may be connected through a channel through which fluid may flow. When the magnetic particle attached to the magnetic immune particle are moved toward the magnet of the magnetic field forming unit by the magnetic field formed by the magnetic field forming unit, the magnetic immune particle may be isolated from the sample. The isolated magnetic immune particle may include a magnetic immune particle that have bound to a substance to be captured in the sample, as well as a magnetic immune particle that have failed to bind to any substance. Thus, both the substance to be captured and the magnetic immune particle may be removed from the sample in the magnetic field forming unit.

There is no particular limitation on the shape of the integrated or separately provided reaction unit and/or magnetic field forming unit, and may have various shapes such as a well shape, a plate shape, or a channel shape, etc. There is no particular limitation on the number of the integrated or separately provided reaction unit and/or magnetic field forming unit, and each may be one or more. For example, if the reaction unit and magnetic field forming unit are separately provided, there may be one or more, for example, 1 to 10 reaction units connected with 1 to 10 magnetic field forming units, or if the reaction unit and magnetic field forming unit form an integral unit, there may be 1 to 10 reaction units and magnetic field forming units, but is not limited thereto.

The kit or device may include one or more injection units connected to the reaction unit, into which a sample (in a fluid such as blood, etc.), magnetic immune particle (for example, in a dispersion), other additional component (for example, opsonin, blood or plasma other than the sample, etc.), or reactant thereof, are injected. The other side of the injection unit not connected to the reaction unit of the injection unit may be directly connected to the subject, or may be connected to an isolated sample, whereby the sample may be injected through the injection unit. Furthermore, the magnetic immune particle may be injected through the other side of the injection unit not connected to the reaction unit of the injection unit. In addition, additional components, such as opsonin, blood or plasma, or the like, other than the sample, may be injected through the other side of the injection unit that is not connected to the reaction unit of the injection unit. The injection unit may include one or more pumps or may be connected to one or more pumps. One or more selected from among the sample, magnetic immune particle, other additional component, and reactant thereof may be injected into the reaction unit through the injection unit by the pump.

The kit or device may additionally include one or more discharge units connected to the magnetic field forming unit that discharge the magnetic immune particle captured by the magnetic field. The magnetic immune particle isolated from the sample discharged through the discharge unit may include a magnetic immune particle that have bound to a substance to be captured in the sample, as well as a magnetic immune particle that have failed to bind to any substance. The discharge unit may further include a detection unit that provide means of detection capable of detecting substances captured by the discharged magnetic immune particle.

In the kit or device, in the magnetic field forming unit, the magnetic immune particle may be separated from the sample by a magnetic field and collected or concentrated separately. The magnetic immune particle may include all magnetic immune particles that have or have not captured the substance to be captured. In this case, the magnetic immune particle may not be discharged, but may be filtered, collected, concentrated, or removed from within the magnetic field forming unit or from a collection unit connected to magnetic field forming unit.

The kit or device may further include a sample discharge unit.

The sample discharge unit may refer to a portion from which an isolated sample is discharged by applying a magnetic field to the sample. There may be one or more sample discharge units. In the presence of a flow of fluid, a sample that has been moved to a location and isolated by the application of a magnetic field, for example a sample from which the magnetic immune particle have been removed, may be discharged through the sample discharge unit for further concentration or isolation. The magnetic immune particle may include all magnetic immune particles that have or have not captured the substance to be captured. Accordingly, the sample discharged through the sample discharge unit may have all substance to be captured and magnetic immune particle removed.

In the kit or device, the sample discharge unit may be connected to the injection unit, or may be connected to a subject. Therefore, by repeating the process of removing the substance to be captured in the sample by injecting the sample from which the magnetic immune particle have been removed, discharged through the sample discharge unit, again through the injection unit, the substance to be captured that has not been completely removed may be more effectively removed. Additionally, the sample discharged through the sample discharge unit from which the magnetic immune particle have been removed may be reinjected into the subject. The magnetic immune particles may include all magnetic immune particles that have captured or have not captured a substance to be captured. Accordingly, the sample injected back into the subject may have both the substance to be captured and the magnetic immune particle removed.

Another aspect provides a method of preparing the magnetic immune particle, including the phases of mixing magnetic particle with erythrocytes or cell membranes isolated from erythrocytes; and phase of extruding and/or sonicating the mixture obtained in the mixing phase.

The magnetic particle may be a monodisperse magnetic particle. The monodisperse magnetic particle may have a polydispersity index of about 0.17 or less.

The method of preparing the cell membrane, the magnetic particle, the magnetic immune particle, the monodisperse magnetic particle, the polydispersity index, and the magnetic immune particle are as described above.

The method of isolating the cell membrane from the erythrocytes is not particularly limited and may be performed by a general method known in the art. For example, a cell membrane isolated from an erythrocyte may be obtained by hypo-osmotic treatment of a solution including erythrocytes (further centrifugation may be performed after hypo-osmotic treatment), but is not limited thereto. In addition, the process of treating the cell membrane isolated from the erythrocyte with ultrasound at about 10 to 30 KHz and about 100 to 200 W for about 1 to 30 minutes may be further performed, but is not limited thereto.

The mixing may be mixing a solution (for example, suspension) including an erythrocyte or cell membrane isolated from the erythrocyte with a solution (for example, suspension) including the magnetic particle. Thus, the mixture may be a solution (for example, suspension) including the erythrocyte or cell membrane isolated from the erythrocyte, and the magnetic particle.

Extruding the mixture may be performed, for example, by passing the mixture under pressure through one or more filters having a pore size of about 0.05 to 1 μm, but is not limited thereto, the extrusion may be performed by an extruder, extrusion equipment, extrusion kit, and the like generally known in the art.

Sonication of the mixture may be performed by a method and condition generally known in the art, and sonication of the mixture without extrusion may be sufficient to bind or attach the magnetic particle to the erythrocyte or cell membrane isolated from erythrocytes present in the mixture, or to incorporate the magnetic particle into the erythrocyte or cell membrane isolated from erythrocytes.

Extruding or sonicating the mixture may optionally be performed alone or both processes may be performed, and when both processes are performed, the two processes may be performed simultaneously or sequentially without limitation in order.

Through the extruding and/or sonicating the mixture may be to obtain the magnetic immune particle, wherein the erythrocyte or cell membrane isolated from the erythrocyte is bound or attached to one or more of the magnetic particle, or wherein the erythrocyte or cell membrane isolated from the erythrocyte is embedded with one or more of the magnetic particle.

Among the terms or elements mentioned in the methods, kits, and devices, those mentioned in the description of the magnetic immune particles and compositions are understood to be the same as previously mentioned in the description of the magnetic immune particles and compositions.

### Advantageous Effects

The erythrocyte-derived magnetic immune particle according to an aspect include an erythrocyte-derived cell membrane, which may minimize in vivo side effects, and may detect and remove various type of substances (for example, pathogenic substance, inflammatory cytokine, blood glucose, cancer-related substance, and brain disease-related substance) from a sample with excellent efficiency due to the characteristics of the erythrocytes from which the cell membrane is derived. Furthermore, since the magnetic immune particle include magnetic particle, the magnetic immune particle may be simply isolated by applying a magnetic field, thereby enabling more effective detection and removal of the above various types of substance, and when the magnetic immune particle are used for treatment, the possibility of injecting the magnetic immune particle into the body may be minimized, thereby significantly reducing in vivo side effects. As a result, the magnetic immune particle may be useful in diagnosing, preventing, or treating various type of diseases, including an infectious disease, inflammatory disease, diabetes, cancer, and brain disease, and the like.

### DESCRIPTION OF DRAWINGS

FIG. 1 is a schematic diagram illustrating a method of producing a magnetic immune particle, according to an example.

FIG. 2 is a transmission electron micrograph (TEM) illustrating a magnetic immune particle generated according to an example. The left side illustrates a magnetic particle used in an example, and the right side illustrates the magnetic immune particle produced according to an example.

FIG. 3 is a diagram illustrating the capture ability (detection or removal ability) of magnetic immune particle for pathogenic bacteria (A: MRSA and B: ESBL(+) E. coli) according to an example (HL60 (N): neutrophil-derived magnetic immune particle; U937 (M): macrophage-derived magnetic immune particle; hHSEC: human liver endothelial cell-derived magnetic immune particle; hRBC-MNVs: erythrocyte-derived magnetic immune particle).

FIG. 4 is a diagram illustrating the capture ability (detection or removal ability) of magnetic immune particle for viruses (A: CMV and B: RSV) according to an example (HL60 (N)): neutrophil-derived magnetic immune particle; U937 (M): macrophage-derived magnetic immune particle; hHSEC: human liver endothelial cell-derived magnetic immune particle; hRBC-MNVs: erythrocyte-derived magnetic immune particle).

FIG. 5 is a diagram illustrating the capture ability (detection or removal ability) of magnetic immune particle to virus-derived antigenic proteins (A: ZIKV E Protein and B: SARS-COV-2 S Protein) according to an example (HL60 (N)): neutrophil-derived magnetic immune particle; U937 (M): macrophage-derived magnetic immune particle; hHSEC: human liver endothelial cell-derived magnetic immune particle; hRBC-MNVs: erythrocyte-derived magnetic immune particle).

FIG. 6 is a schematic diagram illustrating a magnetic immune particle-based extracorporeal circulation device according to an example.

FIG. 7 is a graph illustrating the results of removing antibiotic-resistant and susceptible bacteria (MRSA, VISA, S. aureus, ESBL(+) E. coli, Carbapenem-resistant E. coli, E. coli) present in a sample through an extracorporeal circulation device based on erythrocyte-derived magnetic immune particle according to an example.

FIG. 8 is a graph illustrating the results of removing endotoxin (LPS) present in a sample via an erythrocyte-derived magnetic immune particle-based extracorporeal circulation device according to an example.

FIG. 9 is a schematic diagram illustrating a magnetic immune particle-based extracorporeal circulation device connected to an animal model according to an example.

FIG. 10 is a graph illustrating the results of removing pathogenic substance from the in vivo blood of a pathogenic bacteria-infected animal using an erythrocyte-derived magnetic immune particle-based extracorporeal circulation device according to an example (A: MRSA level of removal from the in vivo blood of an MRSA-infected animal; B: carbapenem-resistant E. coli level of removal present in the in vivo blood of a carbapenem-resistant E. coli infected animal; C: endotoxin (LPS) level of removal present in the in vivo blood of a carbapenem-resistant E. coli infected animal).

FIG. 11 is a graph illustrating the survival rate of the infected animal as a result of removal of pathogenic substance present in the in vivo blood of pathogenic bacteria-infected animals using an erythrocyte-derived magnetic immune particle-based extracorporeal circulation device according to an embodiment (A: survival rate of MRSA-infected animals; B: survival rate of Carbapenem-resistant E. coli-infected animals).

FIG. 12 is a graph illustrating the results of removing interleukin 6 (IL-6) present in a sample via an erythrocyte-derived magnetic immune particle-based extracorporeal circulation device according to an embodiment.

FIG. 13 is a graph illustrating the results of removing pro-inflammatory cytokines present in the in vivo blood of an animal infected with a pathogenic bacteria using an erythrocyte-derived magnetic immune particle-based extracorporeal circulation device according to an embodiment (A: level of removal of pro-inflammatory cytokines present in the in vivo blood of an MRSA-infected animal; B: level of removal of pro-inflammatory cytokines present in the in vivo blood of a Carbapenem-resistant E. coli-infected animal).

FIG. 14 is a diagram illustrating the capture ability (detection or removal ability) of erythrocyte-derived magnetic immune particle according to an embodiment for blood glucose in a blood sample (A) and the capture ability (detection or removal ability) for pathogenic bacteria in a hyperglycemic blood sample (B).

FIG. 15 is a diagram illustrating the decrease in capture ability (detection or removal ability) of various pathogens of erythrocyte-derived magnetic immune particle inactivated with cell membrane surface molecules (CR1 and/or GYPA) according to an embodiment (RBC-MNVs: erythrocyte-derived magnetic immune particle; CR1 blocked RBC-MNVs: erythrocyte-derived magnetic immune particle with CR1 inactivated; GYPA blocked RBC-MNVs: erythrocyte-derived magnetic immune particle with GYPA inactivated; GYPA&CR1 blocked RBC-MNVs: erythrocyte-derived magnetic immune particle with GYPA and CR1 inactivated; MNPs: Magnetic particle that do not include a cell membrane surface).

FIG. 16A is a diagram illustrating the results of an analysis of the capture ability (detection or removal ability) of erythrocyte-derived magnetic immune particle according to an embodiment for the pathogenic substance (pathogenic bacteria) when injected together with opsonin into a sample (TBS buffer) including the pathogenic substance.

FIG. 16B is a diagram illustrating the results of an analysis of the capture ability (detection or removal ability) of the erythrocyte-derived magnetic immune particle for the pathogenic substance according to an embodiment when injected together with opsonin into a sample (TBS buffer) including a pathogenic substance (virus or virus-derived antigenic protein).

FIG. 16C is a diagram illustrating the results of an analysis of the capture ability (detection or removal ability) of erythrocyte-derived magnetic immune particle according to an embodiment for the pathogenic substance (pathogenic bacteria) when injected together with opsonin into a sample (blood sample) including the pathogenic substance.

FIG. 17 is a diagram illustrating the capture ability (detection or removal ability) of erythrocyte-derived magnetic immune particle according to an embodiment for a cancer-related substance (cancer cell line-derived extracellular vesicle) present in a human plasma and blood sample.

FIG. 18 is a diagram illustrating the capture ability (detection or removal ability) of an erythrocyte-derived magnetic immune particle for cancer-related substance (tumor cell-derived nucleic acid) according to an embodiment (A: capture ability (detection or removal ability) of erythrocyte-derived magnetic immune particle for nucleic acid derived from normal cells and nucleic acid derived from tumor cells; B: capture ability (detection or removal ability) of human erythrocyte-derived magnetic immune particle and mouse erythrocyte-derived magnetic immune particle for tumor cell-derived nucleic acid).

FIG. 19 is a graph illustrating the recovery rate (A) and purity (B) of the recovered nucleic acid by an elution solution of nucleic acids derived from normal cells and nucleic acids derived from tumor cells captured by erythrocyte-derived magnetic immune particle according to an embodiment.

FIG. 20 is a diagram illustrating the capture ability (detection or removal ability) of erythrocyte-derived magnetic immune particle according to an embodiment for a substance (amyloid beta 42) related with brain disease (Alzheimer's disease).

FIG. 21 is a graph illustrating the results of removing brain disease (Alzheimer's disease) related substances (A: tau protein; B: amyloid beta 40; C: amyloid beta 42) present in a sample via an erythrocyte-derived magnetic immune particle-based extracorporeal circulation device according to an embodiment.

FIG. 22 is a graph illustrating the diameter of monodisperse magnetic particle and polydisperse magnetic particle according to an embodiment.

FIG. 23 is a diagram illustrating the capture ability (detection or removal ability) of monodisperse magnetic immune particle and polydisperse magnetic immune particle according to an embodiment for cancer-related substances (A: nucleic acid derived from circulating tumor cells; B: extracellular vesicles derived from cancer cell lines).

FIG. 24 is a diagram illustrating the capture ability (detection or removal ability) of brain disease (Alzheimer's disease) related substances (amyloid beta 40, amyloid beta 42, tau protein) of monodisperse magnetic immune particle and polydisperse magnetic immune particle according to an embodiment.

### MODE FOR INVENTION

The present disclosure will be described in more detail below with reference to embodiments. However, these embodiments are intended to illustrate the present disclosure by way of example and the scope of the invention is not limited to these embodiments.

### Example 1. Preparation of Erythrocyte-Derived Magnetic Immune Particle

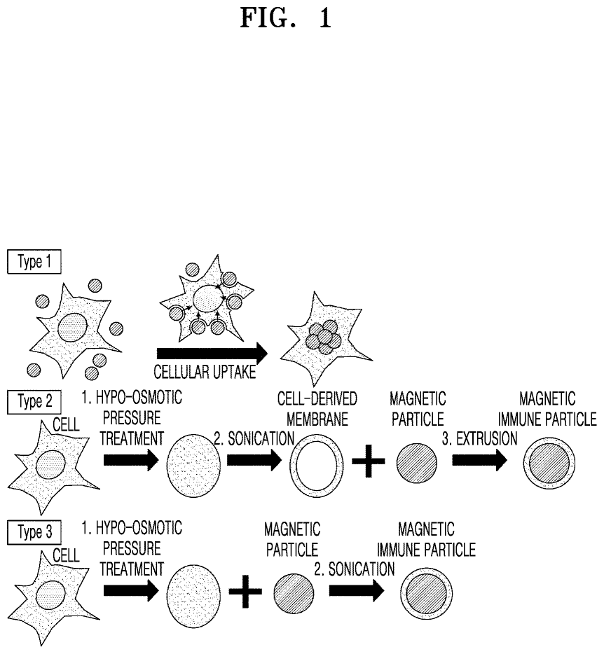

Magnetic immune particles were prepared using a model of human erythrocytes in human in vivo blood. Erythrocytes were obtained from the Red Cross (Red Cross, South Korea) and blood from donors who are willing to participate (UNISTIRB-20-44-A). Erythrocytes were prepared in 1×PBS by inoculating about 10⁶ cells in about 1 mL of a mixture of about 25% v/v of PBS (pH 7.2, Biosesang, South Korea) and distilled water (Biosesang, South Korea), followed by hypo-osmotic treatment at 4° C. for 1 hour and centrifugation at 4° C. for 5 minutes (Centrifuge 5424R, Eppendorf, Germany). In addition, the cell membranes isolated (purified) by hypo-osmotic treatment were sonicated (Q700 Ultra-Sonicator, Qsonica, USA) at 4° C., 20 KHz, 150 W for about 10 minutes to break the cell membranes into smaller units. Afterwards, the sonicated erythrocyte-derived cell membrane was extruded with magnetic particle in an Avanti mini extruder (Avanti Polar Lipids, Alabaster, AL, USA) using 1 μm, 0.4 μm, and 0.2 μm pore size track-etched membrane filters to prepare magnetic immune particle. Specifically, as in the type 2 method shown in FIG. 1, the prepared magnetic immune particles were prepared by mixing and extruding the above-prepared cell membrane with magnetic particle (about 0.5 mg/mL), and embedding the magnetic particle inside the cell membrane. As a magnetic particle, iron oxide magnetic particle (Carboxyl-Adembeads 200 nm, Ademtech, France) with an average particle diameter of about 100 to 300 nm (specifically, about 200 nm) that has carboxylic acid modified on the surface were used.

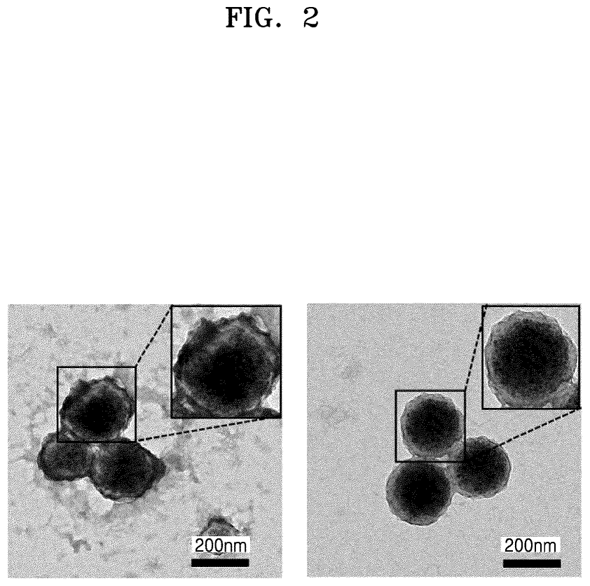

As a result, as shown in FIG. 2, it was confirmed by transmission electron micrograph (TEM) imaging that magnetic immune particle (right side of FIG. 2) with an average diameter of about 150 to 350 nm (specifically, about 250 nm) were generated, in which the magnetic particles were incorporated into the erythrocyte-derived cell membrane.

### Experimental Example 1. Evaluation of Capture Ability (Detection or Removal Ability) of Erythrocyte-Derived Magnetic Immune Particle Against Pathogenic Substance

To determine whether the erythrocyte-derived magnetic immune particle prepared in Example 1 may capture (detect or remove) various pathogenic substances including pathogens, etc., present in the blood, a human blood sample was randomly inoculated with a pathogenic substance, the erythrocyte-derived magnetic immune particle was injected, and a magnetic field was applied to measure changes in the concentration of the pathogenic substance in the blood sample.

### 1.1. Evaluation of Capture Ability (Detection or Removal Ability) of Erythrocyte-Derived Magnetic Immune Particle Against Pathogenic Bacteria

The capture ability (detection or removal ability) of the erythrocyte-derived magnetic immune particle against pathogenic bacteria was evaluated.

Specifically, about 1 mL of anticoagulated human blood (Red Cross, South Korea) sample was inoculated with either MRSA (Methicillin Resistant Staphylococcus aureus), a Gram-positive bacterium, or ESBL(+) E. coli (Extended-Spectrum Beta-Lactamases Producing Escherichia coli), a Gram-negative bacterium, at a concentration of about 10⁴ CFU/mL, and incubated at about 37° C. for about 10 minutes. At the end of the incubation, the blood sample was injected with the erythrocyte-derived magnetic immune particle prepared in Example 1 above, such that the concentration of the magnetic immune particle was finally about 100 to 200 μg/mL. Thereafter, after a reaction of about 20 minutes at about 37° C., the magnetic immune particles in the blood sample were fixed at a specific position using a magnet to prevent the magnetic immune particle from being included in the supernatant, and the supernatant was collected to determine the colony forming portion (CFU) of the bacteria in the supernatant. Specifically, the supernatant (about 100 μL) was diluted in about 900 μL of saline solution and smeared on LB agar medium using a microbial analyzer (EDDY JET2, IUL micro, USA), cultured at about 37° C. for about 24 hours, and the CFU of bacteria formed on the LB agar medium was measured using a microbial colony counter (Sphereflash colony counter and zone reader, IUL micro, USA). A blood sample was inoculated with the above bacteria and cultured in the same method as described above, and a sample not injected with the above erythrocyte-derived magnetic immune particle was used as a control group. Based on the CFU value of the bacteria measured in the control group, the level of reduction of the CFU value of the bacteria measured in the experimental group was calculated as a percentage (%), and the capture rate (or removal rate, %) of the erythrocyte-derived magnetic immune particle on the bacteria was evaluated. In addition, neutrophil-derived magnetic immune particle utilizing neutrophil (HL60)-derived cell membrane; macrophage-derived magnetic immune particle utilizing macrophage (U937)-derived cell membrane; and human liver endothelial cell-derived magnetic immune particle utilizing human liver endothelial cell (hHSEC)-derived cell membrane, prepared by the same method as the method of Example 1, were used as a comparison group.

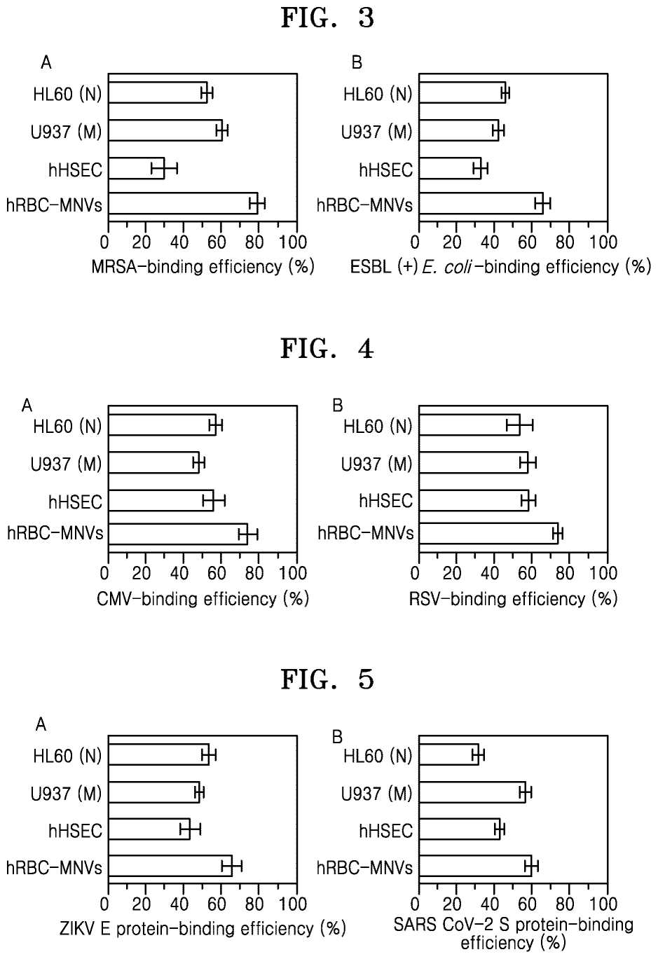

As a result, as shown in FIG. 3, it was confirmed that the erythrocyte-derived magnetic immune particle prepared in Example 1 may capture and detect or remove pathogenic bacteria in the blood sample, and was confirmed to exhibit a remarkably excellent capture rate (about 60 to 90%) even for antibiotic-resistant bacteria such as MRSA and ESBL(+) E. coli. In particular, it was confirmed that compared to the neutrophil-derived magnetic immune particle, the macrophage-derived magnetic immune particle, and the human liver endothelial cell-derived magnetic immune particle, the erythrocyte-derived magnetic immune particle were found to have better capture ability (detection or removal ability) for pathogenic bacteria such as MRSA and ESBL(+) E. coli.

### 1.2. Evaluation of Capture Ability (Detection or Removal Ability) of Erythrocyte-Derived Magnetic Immune Particle Against Virus

The capture ability (detection or removal ability) of the erythrocyte-derived magnetic immune particle against a virus was evaluated.

Specifically, about 1 mL of anticoagulated human blood (Red Cross, South Korea) sample was inoculated with cytomegalovirus (CMV) or respiratory syncytial virus (RSV) at a concentration of about 10⁴ PFU/mL and incubated at about 37° C. for about 10 minutes. At the end of the incubation, the blood sample was injected with the erythrocyte-derived magnetic immune particle prepared in Example 1, such that the concentration of the magnetic immune particle was finally about 200 μg/mL. Thereafter, after a reaction of about 20 minutes at about 37° C., the magnetic immune particle in the blood sample were fixed at a specific position using a magnet to prevent the magnetic immune particle from being included in the supernatant, and the supernatant was collected to measure the amount of RNA of the virus in the supernatant. Nucleic acid was extracted from the virus present in the supernatant using the QIAmp viral RNA mini kit (QIAGEN, Germany), and the extracted nucleic acid was amplified using SYBR PCR master mix (Toyobo, Japan) and Real time PCR (CFX connect, BIO-RAD, USA) to measure the amount of RNA. A blood sample was inoculated with the above virus and cultured in the same method as described above, and a sample not injected with the above erythrocyte-derived magnetic immune particle was used as a control group. Based on the amount of RNA of the virus measured in the control group, the level of reduction of the amount of RNA of the virus measured in the experimental group was calculated as a percentage (%), and the capture rate (or removal rate, %) of the erythrocyte-derived magnetic immune particle on the virus was evaluated. In addition, neutrophil-derived magnetic immune particle utilizing neutrophil (HL60)-derived cell membrane; macrophage-derived magnetic immune particle utilizing macrophage (U937)-derived cell membrane; and human liver endothelial cell-derived magnetic immune particle utilizing human liver endothelial cell (hHSEC)-derived cell membrane, prepared by the same method as the method of Example 1, were used as a comparison group.

As a result, as shown in FIG. 4, it was confirmed that the erythrocyte-derived magnetic immune particle prepared in Example 1 may capture and detect or remove pathogenic virus in the blood sample (capture rate: about 60 to 80%). In particular, it was confirmed that compared to the neutrophil-derived magnetic immune particle, the macrophage-derived magnetic immune particle, and the human liver endothelial cell-derived magnetic immune particle, the erythrocyte-derived magnetic immune particle were found to have better capture ability (detection or removal ability) for pathogenic virus such as CMV and RSV.

### 1.3. Evaluation of Capture Ability (Detection or Removal Ability) of Erythrocyte-Derived Magnetic Immune Particle Against Virus-Derived Antigen

The capture ability (detection or removal ability) of the erythrocyte-derived magnetic immune particle against a virus-derived antigen was evaluated.

Specifically, about 1 mL of anticoagulated human blood (Red Cross, South Korea) sample was inoculated with Zika virus (ZIKV) Envelope Protein (ZIKV E Protein) or SARS-COV-2 Spike Protein (SARS-COV-2 S Protein) at a concentration of about 1 μg/mL, and erythrocyte-derived magnetic immune particle prepared in Example 1 above were injected at a concentration of about 200 μg/mL. Thereafter, the magnetic immune particle in the blood sample were fixed at a specific position using a magnet to prevent the magnetic immune particle from being included in the supernatant, and the supernatant was collected to measure the concentration of the virus-derived antigen in the supernatant. The concentration of the virus-derived antigen was measured by enzyme-linked immunosorbent assay (ELISA), and the Zika virus (strain Zika SPH2015) Envelope Protein (ZIKV-E) ELISA Kit (Sinobio, China) or SARS-COV-2 Spike protein ELISA kit (ab274342, abcam, USA) was used for measurement. The capture rate (or removal rate, %) of the erythrocyte-derived magnetic immune particle for the virus-derived antigen was evaluated by calculating the level of decrease in virus-derived antigen concentration measured in the supernatant as a percentage (%), based on the concentration of virus-derived antigen in the blood sample before injection of the magnetic immune particle. In addition, neutrophil-derived magnetic immune particle utilizing neutrophil (HL60)-derived cell membrane; macrophage-derived magnetic immune particle utilizing macrophage (U937)-derived cell membrane; and human liver endothelial cell-derived magnetic immune particle utilizing human liver endothelial cell (hHSEC)-derived cell membrane, prepared by the same method as the method of Example 1, were used as a comparison group.

As a result, as shown in FIG. 5, it was confirmed that the erythrocyte-derived magnetic immune particle prepared in Example 1 may capture and detect or remove virus-derived antigenic protein in the blood sample (capture rate: about 50 to 70%). In particular, it was confirmed that compared to the above neutrophil-derived magnetic immune particle, the above macrophage-derived magnetic immune particle, and the above human liver endothelial cell-derived magnetic immune particle, the above erythrocyte-derived magnetic immune particle have better capture ability (detection or removal ability) for virus-derived antigenic protein such as ZIKV E Protein and SARS-CoV-2 S Protein.

### 1.4. Evaluation of Capture Ability (Detection or Removal Ability) of Erythrocyte-Derived Magnetic Immune Particle for 137 Species of Bacteria Found in Fecal Samples Used for Fecal Microbiota Transplantation (FMT)

The capture ability (detection or removal ability) of the erythrocyte-derived magnetic immune particle prepared in Example 1 above against 137 species of bacteria (see Table 1) found in fecal samples used for fecal microbiota transplantation (FMT) was evaluated using the same method as in Experimental Example 1.1 above, and the results are shown in Table 1 below.

TABLE 1

| | Removal | | Removal |
|---|---|---|---|
| Bacteria | rate (%) | Bacteria | rate (%) |
| Fusobacterium perfoetens | 99.99014 | Blautia argi | 99.76875 |
| Anaerobiospirillum | 100 | Blautia obeum | 99.99168 |
| succiniciproducens | | | |
| Phocaeicola plebeius | 99.98403 | Anaerotignum faecicola | 99.99667 |
| Mediterranea massiliensis | 99.97913 | Flavonifractor plautii | 99.97116 |
| Fusobacterium mortiferum | 99.95149 | Collinsella intestinalis | 99.81064 |
| Faecalibacterium prausnitzii | 99.96941 | Bacteroides cellulosilyticus | 98.33818 |
| Mucispirillum schaedleri | 99.99316 | Allobaculum stercoricanis | 99.93248 |
| Phocaeicola coprophilus | 99.99233 | Fournierella massiliensis | 99.96307 |
| Brachyspira hampsonii | 100 | Haemophilus haemoglobinophilus | 99.73614 |
| Bacteroides uniformis | 99.9847 | Hungatella xylanolytica | 99.98594 |
| Phocaeicola coprocola | 99.9615 | Ruminococcus lactaris | 99.94898 |
| Phocaeicola vulgatus | 99.97373 | Other | 99.97268 |
| Corynebacterium amycolatum | 100 | Catenibacterium mitsuokai | 97.99739 |
| Sutterella massiliensis | 99.8365 | Clostridium methylpentosum | 99.96376 |
| Lachnospira eligens | 99.97393 | Flintibacter butyricus | 99.98363 |
| Lachnoclostridium pacaense | 99.98789 | Peptostreptococcus canis | 99.96395 |
| Corynebacterium lowii | 100 | Pasteurella stomatis | 100 |
| Prevotella stercorea | 99.9932 | Mediterraneibacter | 99.92791 |
| | | glycyrrhizinilyticus | |
| Blautia marasmi | 99.94649 | Helicobacter winghamensis | 100 |
| Peptacetobacter hiranonis | 99.84061 | Holdemanella biformis | 98.94652 |
| Lachnospira pectinoschiza | 99.98531 | Bacteroides caccae | 99.98326 |
| Kineothrix alysoides | 99.96107 | Dialister invisus | 99.98326 |
| Bacteroides thetaiotaomicron | 99.97833 | Enterococcus gallinarum | 100 |
| Lactobacillus rogosae | 99.9878 | Blautia schinkii | 98.73838 |
| Eubacterium rectale | 99.97145 | Blautia wexlerae | 100 |
| Paraprevotella clara | 99.9821 | Pseudoflavonifractor phocaeensis | 100 |
| Ruminococcus gnavus | 99.93576 | Desulfovibrio simplex | 100 |
| Roseburia intestinalis | 99.97928 | Schaalia canis | 99.96615 |
| Pseudomonas matsuisoli | 99.98258 | Amedibacillus dolichus | 99.96264 |
| Corynebacterium confusum | 100 | Turicibacter sanguinis | 99.76724 |
| Megamonas funiformis | 98.34063 | Enterococcus faecalis | 100 |
| Bacteroides stercoris | 99.98891 | Monoglobus pectinilyticus | 99.95674 |
| Helicobacter canicola | 100 | Agathobaculum butyriciproducens | 99.96692 |
| Winkia neuii | 100 | Staphylococcus simulans | 100 |
| Aerococcus vaginalis | 100 | Enterococcus dispar | 100 |
| Roseburia inulinivorans | 99.98123 | Blautia luti | 100 |
| Porphyromonas cangingivalis | 99.86023 | Hespellia porcina | 100 |
| Bacteroides koreensis | 99.98575 | Helicobacter bilis | 100 |
| Roseburia hominis | 99.99708 | Prevotella copri | 46.55614 |
| Sutterella stercoricanis | 99.99581 | Caproiciproducens | 99.90683 |
| | | galactitolivorans | |
| Gemmiger formicilis | 99.97684 | Romboutsia sedimentorum | 99.9634 |
| Alistipes putredinis | 99.98396 | Dorea formicigenerans | 100 |
| Parabacteroides merdae | 99.98566 | Klebsiella variicola | 100 |
| Blautia faecis | 99.96416 | Tyzzerella nexilis | 99.66837 |
| Eubacterium ventriosum | 99.99581 | Holdemania massiliensis | 99.98198 |
| Butyricicoccus pullicaecorum | 99.964 | Candidatus Pelagibacter ubique | 100 |
| Faecalimonas umbilicata | 99.93603 | Bifidobacterium catenulatum | 99.93315 |
| Escherichia fergusonii | 99.96595 | Campylobacter showae | 99.97247 |
| Staphylococcus felis | 100 | Erysipelatoclostridium ramosum | 99.98034 |
| Roseburia faecis | 99.9843 | Clostridium tertium | 99.9827 |
| Bacteroides pyogenes | 99.93792 | Peptococcus niger | 99.9243 |
| Helicobacter canis | 100 | Buchananella hordeovulneris | 100 |
| Bacteroides fragilis | 99.95284 | Eubacterium coprostanoligenes | 99.8517 |
| Parabacteroides distasonis | 99.98583 | Clostridium paraputrificum | 100 |
| Helicobacter cinaedi | 100 | Eubacterium ramulus | 100 |
| Staphylococcus intermedius | 100 | Dorea longicatena | 100 |
| Anaerostipes hadrus | 99.9976 | Demequina aestuarii | 100 |
| Phascolarctobacterium | 100 | Streptococcus canis | 100 |
| succinatutens | | | |
| Fusicatenibacter saccharivorans | 99.98648 | Lachnospira multipara | 100 |
| Clostridium spiroforme | 99.98289 | Anaerobium acetethylicum | 100 |
| Alistipes shahii | 99.95674 | Anaerobutyricum hallii | 100 |
| Frederiksenia canicola | 100 | Coprococcus catus | 100 |
| Enterocloster clostridioformis | 100 | Nitrososphaera viennensis | 100 |
| Bacteroides xylanisolvens | 99.92538 | Paludibacter propionicigenes | 100 |
| Longibaculum muris | 100 | Synechococcus rubescens | 100 |
| Oscillibacter ruminantium | 99.98621 | Dietzia maris | 100 |
| Corynebacterium auriscanis | 100 | Campylobacter upsaliensis | 100 |
| Agathobaculum desmolans | 100 | Escherichia marmotae | 100 |
| Klebsiella pneumoniae | 100 | | |

As a result, as shown in Table 1, it was confirmed that the erythrocyte-derived magnetic immune particle prepared in Example 1 have a remarkably good capture ability (detection or removal ability) for various types of bacteria, and have a fairly broad spectrum for bacterial capture (detection or removal).

### Experimental Example 2. Confirmation of Removal of Pathogenic Substance in Blood Sample In Vitro Using Extracorporeal Circulation Device Based on Erythrocyte-Derived Magnetic Immune Particle

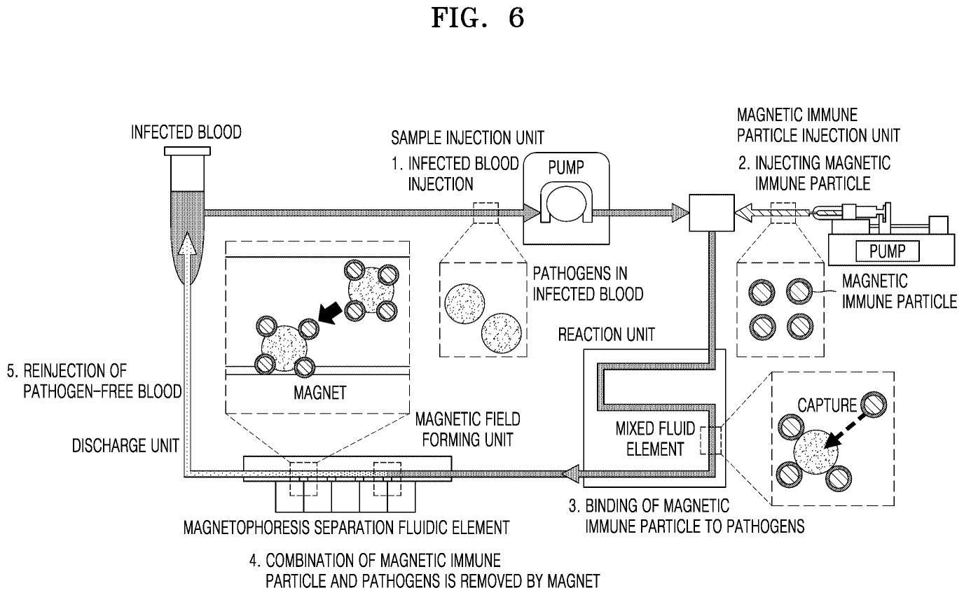

Using the erythrocyte-derived magnetic immune particle-based extracorporeal circulation device, pathogenic substances such as pathogens and endotoxins present in blood samples were removed in vitro. Specifically, as shown in FIG. 6 the erythrocyte-derived magnetic immune particle-based extracorporeal circulation device includes: an injection unit (sample injection unit and magnetic immune particle injection unit) in which blood contaminated with a pathogenic substance and erythrocyte-derived magnetic immune particle are injected; a reaction unit (mixing fluid element portion) in which the injected blood and magnetic immune particle are mixed and the pathogenic substance is captured (or bound) to the magnetic immune particle; a magnetic field forming unit (magnetophoresis separation fluidic element portion) wherein magnetic immune particle with the pathogenic substance captured (or bound) (and magnetic immune particle without the pathogenic substance captured (or bound)) are removed from the blood by a magnetic field; and a discharge unit wherein blood with the pathogenic substance and the magnetic immune particle removed is discharged.

Specifically, in about 1 mL of a sample of anticoagulated human blood (blood from a willing donor, UNISTIRB-20-44-A), was inoculated with a pathogenic substance, pathogenic bacteria (Staphylococcus aureus (S. aureus), MRSA, Vancomycin-intermediate resistant S. aureus (VISA), Escherichia coli (E. coli), ESBL(+) E. coli (ESBL-producing E. coli), or Carbapenem-resistant E. coli; about 10⁴ CFU/mL) or endotoxin (Lipopolysaccharide: LPS; about 10 μg/mL) and cultured at about 37° C. for about 10 minutes. In addition, a saline solution including the erythrocyte-derived magnetic immune particle prepared in Example 1 above at a concentration of about 0.1 to 1 mg/mL was prepared. The cultured blood sample was circulated using an extracorporeal circulation device at a rate of about 10 mL/hr, and the solution including the erythrocyte-derived magnetic immune particle was loaded into the extracorporeal circulation device at a rate of about 0.5 mL/hr. As the injected blood sample and the magnetic immune particle solution flowed through the reaction unit (mixing fluidic element portion) and underwent a mixing process, the pathogenic substances in the blood sample and the magnetic immune particle were bound. While the blood sample including the conjugate passed through the magnetic field forming unit (magnetophoresis separation fluidic element portion), the conjugate (and the magnetic immune particle to which the pathogenic substance was not bound) was captured by the magnet and removed from the blood sample by the magnetic field. The blood sample from which the pathogenic substance and magnetic immune particle were removed was drained and collected, and then injected back into the extracorporeal circulation device. This process of removing pathogenic substances through erythrocyte-derived magnetic immune particle-based extracorporeal circulation was repeated for about 5 hours. At the same time, the concentration of pathogenic substance in the blood sample was measured on an hourly basis. Specifically, the change in the concentration of bacteria in the blood sample was determined by measuring the CFU of bacteria using the same method as in Experimental Example 1.1 above, and the change in the concentration of LPS in the blood sample was determined by enzyme-linked immunosorbent assay (ELISA), using the LPS ELISA kit (LS-F55757-1, LSbio, USA). In addition, an experimental group without injection of the magnetic immune particle was used as a control group.

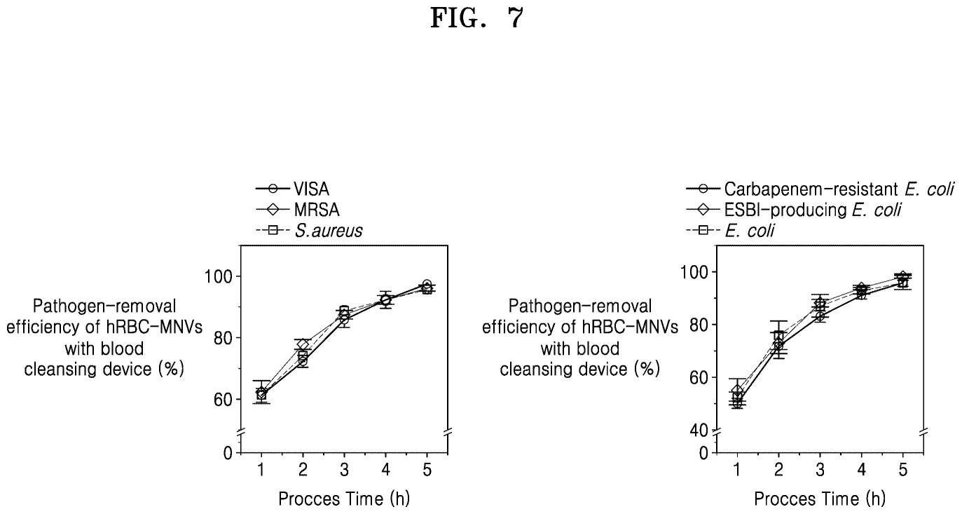

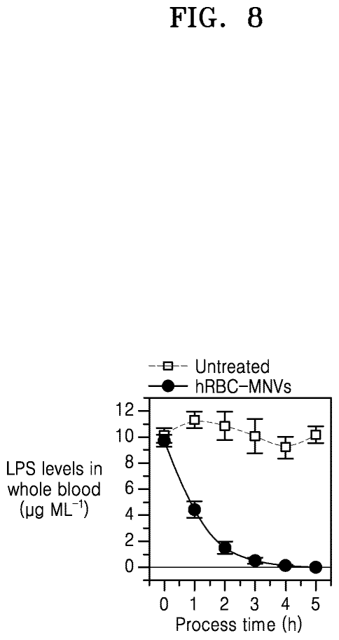

As a result, as shown in FIG. 7 and FIG. 8, it was found that the erythrocyte-derived magnetic immune particle prepared in Example 1 above were capable of very effectively removing pathogenic substance such as pathogens, endotoxin present in samples such as blood, for example antibiotic-resistant and susceptible bacteria (for example, MRSA, VISA, S. aureus, ESBL(+) E. coli, Carbapenem-resistant E. coli, E. coli, etc.) and endotoxin (LPS). Furthermore, it was found that the capture ability (detection or removal ability) of the erythrocyte-derived magnetic immune particle for pathogenic substances is fully exerted even in the case of the above extracorporeal circulation device, and that pathogenic substances such as pathogens, endotoxin, etc., may be sufficiently removed from the sample in vitro. In particular, it was found that in the case of the above extracorporeal circulation device, the process of removing pathogenic substances from the sample using the above erythrocyte-derived magnetic immune particle may be repeated, so that pathogenic substances such as pathogens, endotoxin, etc., present in the sample may be removed by about 90% or more, up to 100%.

### Experimental Example 3. Confirmation of Removal of Pathogenic Substance in Blood In Vivo Using Extracorporeal Circulation Device Based on Erythrocyte-Derived Magnetic Immune Particle and the Effectiveness of Treating Infectious Disease

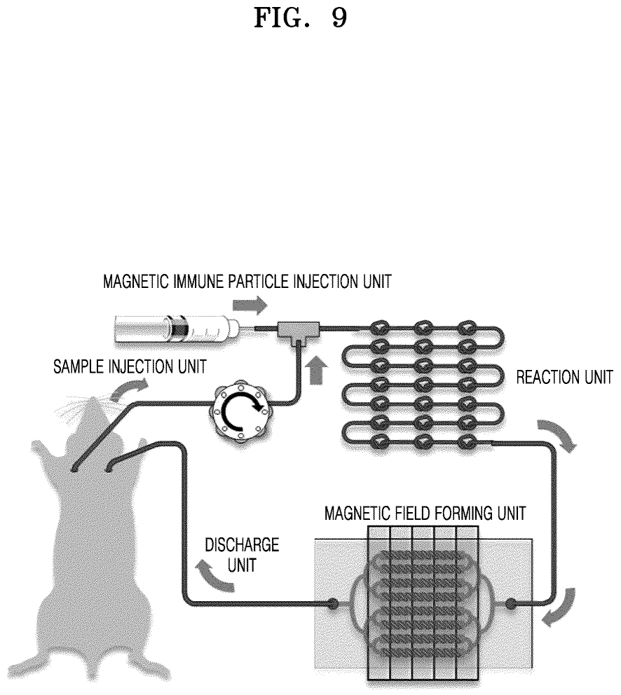

By applying the erythrocyte-derived magnetic immune particle-based extracorporeal circulation device to an animal model, the effect of removing pathogenic substances such as pathogens, endotoxin, etc., present in the blood in vivo and the resulting treatment effect of infectious diseases was confirmed. The method of removing pathogenic substances using the erythrocyte-derived magnetic immune particle-based extracorporeal circulation device is the same as described in Experimental Example 2 above, and differs from Experimental Example 2 above in that in the present experimental example, an in vivo evaluation was performed by directly connecting the erythrocyte-derived magnetic immune particle-based extracorporeal circulation device (see FIG. 9) to a rat animal model infected with pathogenic bacteria. The erythrocyte-derived magnetic immune particle-based extracorporeal circulation device is as described in Experimental Example 2.

Specifically, as shown in FIG. 9, normal Wistar rats (10 weeks old, male) were anesthetized and a catheter was surgically inserted into the jugular vein, and MRSA (about 1×10¹⁰ CFU) or Carbapenem-resistant E. coli (about 5×10⁹ CFU) was randomly injected through the catheter to infect the rat animal. In addition, a magnetic immune particle solution including magnetic immune particle prepared using cell membranes of Wistar rat (8-week-old, male) derived erythrocytes at a concentration of about 0.5 mg/mL was prepared in saline. Through the catheter, the infected rat and the sample injection unit of the erythrocyte-derived magnetic immune particle-based extracorporeal circulation device were connected, and through the sample injection unit, blood of the infected rat was injected into the extracorporeal circulation device. Additionally, the prepared magnetic immune particle solution was injected into the extracorporeal circulation device connected to the rat, and the process of removing the pathogenic substance through the erythrocyte-derived magnetic immune particle-based extracorporeal circulation was performed. The blood from which the pathogenic substance and magnetic immune particle were removed was discharged through the discharge unit of the extracorporeal circulation device and re-injected into the rat connected to the discharge unit. Whole blood was collected from rats at regular time intervals to measure changes in the concentration of MRSA, carbapenem-resistant E. coli, and endotoxin (LPS) in the blood, and the survival rate of rats was measured. Changes in the concentration of bacteria in the blood sample was determined by measuring the CFU of bacteria using the same method as in Experimental Example 1.1 above, and the change in the concentration of LPS in the blood sample was determined by enzyme-linked immunosorbent assay (ELISA), using the LPS ELISA kit (LS-F55757-1, LSbio, USA). In addition, an experimental group injected with an antibiotic (colistin) instead of the magnetic immune particle and an experimental group without injecting the magnetic immune particle were used as a control group.

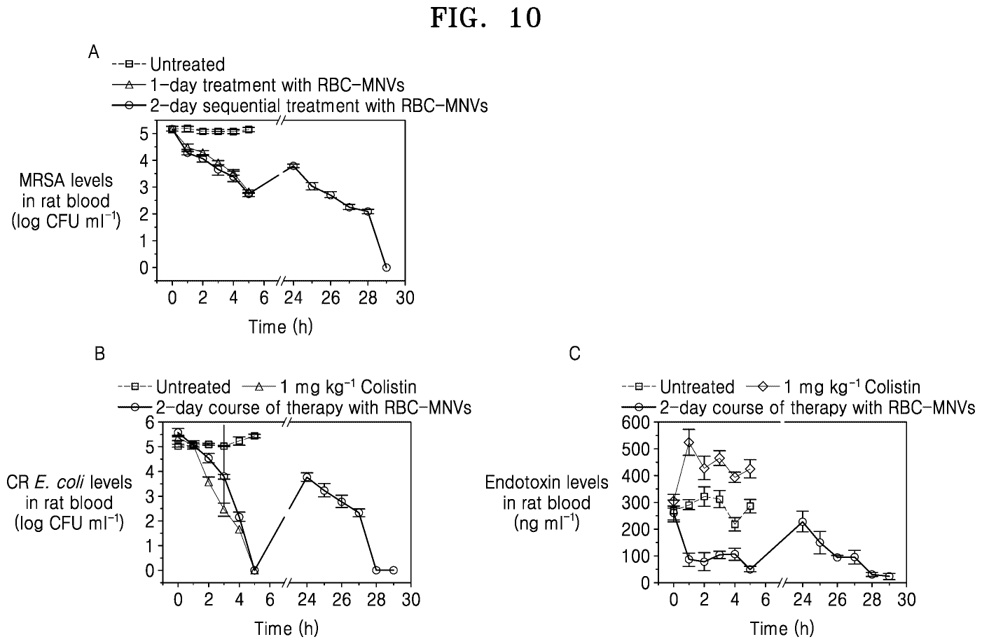

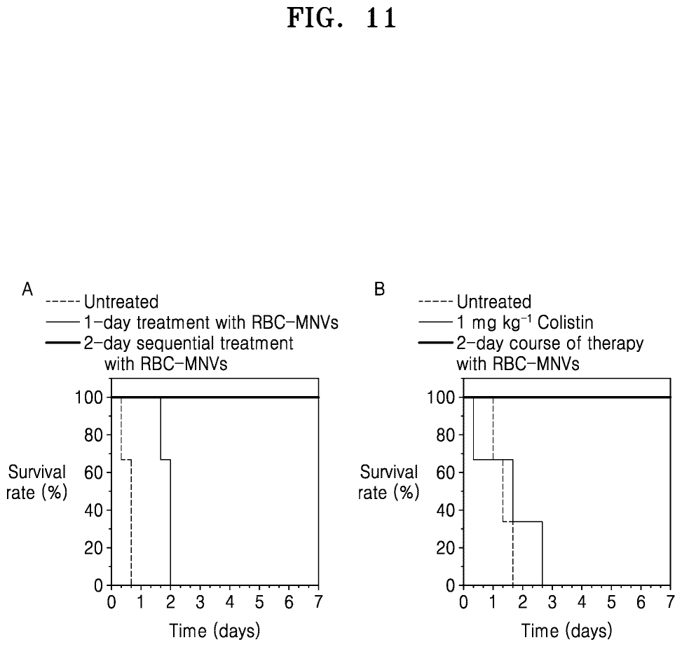

As a result, as shown in FIG. 10, it was confirmed that when erythrocyte-derived magnetic immune particle-based extracorporeal circulation is applied to MRSA or carbapenem-resistant E. coli infected rats through the above extracorporeal circulation device, pathogenic substances such as MRSA, carbapenem-resistant E. coli, and endotoxin (LPS) are very effectively removed from the blood of the infected rats. In particular, it was confirmed that when the erythrocyte-derived magnetic immune particle-based extracorporeal circulation was applied to the infected rats through the above extracorporeal circulation device twice in two days, the concentration of MRSA, carbapenem-resistant E. coli, and endotoxin (LPS) in the blood of the infected rats fell below the measurement limit and the removal efficiency of pathogenic substances was significantly increased. On the other hand, it was confirmed that when an antibiotic (colistin) was injected instead of erythrocyte-derived magnetic immune particle, the concentration of bacteria in the blood of the infected rats could be reduced, but the concentration of endotoxin (LPS) in the blood of the infected rats could not be reduced at all.

Furthermore, as shown in FIG. 11, the survival rate of the infected rats was measured, and it was found that the survival rate of the infected rat was significantly increased when the erythrocyte-derived magnetic immune particle-based extracorporeal circulation was applied through the above extracorporeal circulation device, and in particular, when the erythrocyte-derived magnetic immune particle-based extracorporeal circulation was applied to the infected rat twice in two days, all the infected rats survived for seven days, indicating a 100% survival rate. On the other hand, it was confirmed that in the case of the infected cells that were not injected with erythrocyte-derived magnetic immune particle and the infected cells that were injected with antibiotics (colistin) instead of erythrocyte-derived magnetic immune particle, the survival rate was significantly lower, as all of them died within 3 days.

Through the present experimental example, it was confirmed that an extracorporeal circulation device or method using erythrocyte-derived magnetic immune particle may effectively remove pathogens (for example, pathogenic bacteria or viruses, etc.), endotoxin, and other pathogenic substance present in blood in vivo, thereby treating infectious diseases (for example, pathogenic bacterial or viral infections such as MRSA and carbapenem-resistant E. coli) more effectively than when antibiotics are used.

### Experimental Example 4. Confirmation of Removal of Pro-Inflammatory Cytokine from Blood Using Erythrocyte-Derived Magnetic Immune Particle-Based Extracorporeal Circulation Device

Using an erythrocyte-derived magnetic immune particle-based extracorporeal circulation device, pro-inflammatory cytokine was removed from a blood sample in vitro and from blood in vivo in an animal model of pathogenic bacterial infection. The method of removing pro-inflammatory cytokines from in vitro blood samples and in vivo blood using an erythrocyte-derived magnetic immune particle-based extracorporeal circulation device is the same as described in Experimental Example 2 and Experimental Example 3 above. The erythrocyte-derived magnetic immune particle-based extracorporeal circulation device is as described in Experimental Example 2 and Experimental Example 3 above.

Specifically, in the same method as in Experimental Example 2 above, a blood sample collected from a rat animal (Wistar, male, 8 weeks old) was inoculated with interleukin 6 (Interleukin 6: IL-6), as a pro-inflammatory cytokine, and then cultured, and then, the cultured blood sample and magnetic immune particle prepared using cell membranes of Wistar rat (8-week-old, male)-derived erythrocytes were injected into the extracorporeal circulation device to remove the interleukin 6 by erythrocyte-derived magnetic immune particle-based-extracorporeal circulation. Afterwards, the concentration of interleukin 6 in the discharged blood sample was measured. Furthermore, the experimental group without the injection of the magnetic immune particle was used as a control group.

Furthermore, in the same method as in Experimental Example 3 above, normal Wistar rats were randomly injected with MRSA or Carbapenem-resistant E. coli to infect the rats, and then the infected rats were connected to the above extracorporeal circulation device, and magnetic immune particle prepared using the blood of the infected rats and the cell membrane of Wistar rat (8 week old, male) derived erythrocytes were injected into the above extracorporeal circulation device to remove pro-inflammatory cytokines through erythrocyte-derived magnetic immune particle-based extracorporeal circulation. Afterwards, the discharged blood was re-injected into the rat connected to the extracorporeal circulation device. Before and after performing such erythrocyte-derived magnetic immune particle-based extracorporeal circulation, whole blood from the infected rat was collected and analyzed for pro-inflammatory cytokines in the blood (tumor necrosis factor-α: TNF-α, Interleukin 4 (IL-4), IL-6, Interleukin-1 beta (IL-1), Granulocyte-macrophage colony-stimulating factor (GM-CSF)) were measured in the blood. In addition, an experimental group injected with an antibiotic (colistin) instead of the above magnetic immune particle was used as a control group.

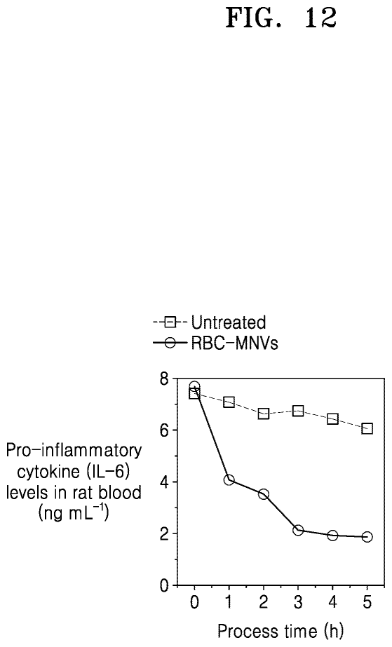

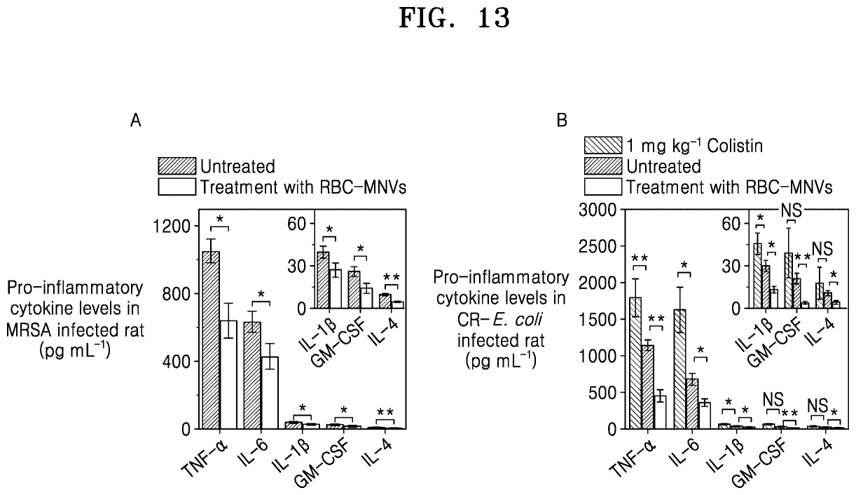

As a result, as shown in FIG. 12 and FIG. 13, it was found that the erythrocyte-derived magnetic immune particles are highly effective in removing pro-inflammatory cytokine present in samples such as blood. In particular, it was found that the erythrocyte-derived magnetic immune particle-based extracorporeal circulation device may very effectively remove not only blood samples in vitro but also pro-inflammatory cytokine present in blood in vivo. Furthermore, it was confirmed that the concentration of pro-inflammatory cytokines in the blood was increased in infected rat injected with antibiotics (colistin) instead of erythrocyte-derived magnetic immune particle, when the erythrocyte-derived magnetic immune particle were used in the extracorporeal circulation device or method, it has been determined that the extracorporeal circulation device or method may remove pro-inflammatory cytokines from the blood more effectively than existing treatment with antibiotics, thereby providing better symptomatic relief (for example, inflammation relief, cytokine storm relief, etc.) or treatment of inflammatory or infectious diseases.

### Experimental Example 5. Evaluation of the Capture Ability (Detection or Removal Ability) Ability of Erythrocyte-Derived Magnetic Immune Particle for Blood Glucose and Pathogenic Substance Present in Hyperglycemic Blood

To determine whether erythrocyte-derived magnetic immune particle may capture (detect or remove) pathogenic substances such as blood glucose and pathogenic bacteria present in hyperglycemic blood, a blood sample with glucose (D-glucose, Sigma-Aldrich, USA) was arbitrarily inoculated with pathogenic bacteria and cultured, and after injecting erythrocyte-derived magnetic immune particle into the culture medium, a magnetic field was applied to measure changes in the concentration of blood glucose and pathogenic bacteria in the blood sample.

Specifically, D-glucose was added to about 1 mL of blood sample collected from a rat animal (Wistar, male, 8 weeks old) to a concentration of about 400 to about 450 mg/dL and cultured at about 37° C. for about 10 minutes. The cultured blood sample was inoculated with MRSA to a concentration of about 10⁴ CFU/mL to 10⁵ CFU/mL and cultured at about 37° C. for about 10 minutes. After cultivation, the blood sample was injected with magnetic immune particle prepared using the cell membrane of Wistar rat (8-week-old, male)-derived erythrocytes, such that the concentration of the magnetic immune particle was finally about 100 to 200 μg/mL. Thereafter, after a reaction of about 20 minutes at about 37° C., the magnetic immune particle in the blood sample were fixed at a specific position using a magnet to prevent the magnetic immune particle from being included in the supernatant, and the supernatant was collected to measure the concentration of D-glucose and MRSA in the supernatant. In addition, the supernatant was reacted by re-injecting the magnetic immune particle into the supernatant, and the process of isolating the magnetic immune particle by a magnet was repeated several times. The change in the concentration of MRSA was confirmed by measuring the CFU of the bacteria using the same method as in Experimental Example 1.1 above, and the change in the concentration of D-glucose was measured using a commercially available blood glucose meter (ACCU-CHEK, Roche, Switzerland). Furthermore, the experimental group without the injection of the magnetic immune particle was used as a control group.

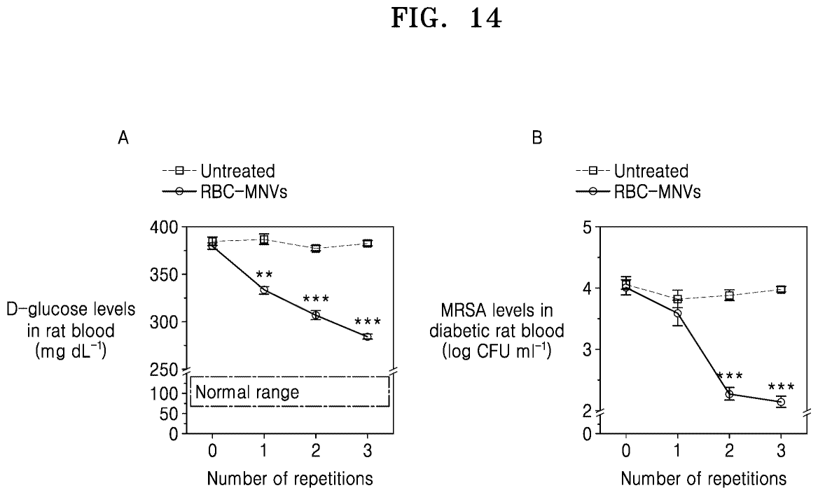

As a result, as shown in FIG. 14, it was confirmed that the concentration of D-glucose and MRSA pathogen in the hyperglycemic blood sample was significantly reduced compared to the control group as the removal process using erythrocyte-derived magnetic immune particle was repeated.

Through the present experimental example it was found that erythrocyte-derived magnetic immune particle exhibit significantly superior blood glucose detection and removal effects, and therefore, erythrocyte-derived magnetic immune particle (for example, an extracorporeal circulation device or method utilizing erythrocyte-derived magnetic immune particle) may be useful for detecting blood glucose in blood in vitro, removing blood glucose by blood purification, and lowering blood glucose in hyperglycemic or diabetic patients by injecting the removed blood back into in vivo. Accordingly, it was found that the erythrocyte-derived magnetic immune particle may be useful in diagnosing, preventing, or treating diabetic disease (for example, alleviating symptoms of diabetic disease). Furthermore, it was found that erythrocyte-derived magnetic immune particle may be highly effective in removing pathogenic substances, such as pathogenic bacteria, present in the blood of hyperglycemic or diabetic patients, and thus may be useful in treating infectious diseases in hyperglycemic or diabetic patients.

### Experimental Example 6. Confirmation of Molecules Present on Surface of Erythrocyte-Derived Magnetic Immune Particle that Contribute to Capture of Pathogenic Substance

To determine which of the various surface molecules, including various receptors, immobilized (attached) to the cell membrane surface of the erythrocyte-derived magnetic immune particle contribute to the capture of pathogenic substances, erythrocyte-derived magnetic immune particle with certain surface molecules inactivated on the cell membrane surface were prepared and the removal rate of pathogenic substances was analyzed.

Specifically, in the erythrocyte-derived magnetic immune particle prepared in Example 1 above, the cell membrane surface molecules CR1 (complement receptor 1) and/or GYPA (glycophorin A) were inactivated with corresponding antibodies, respectively, to obtain erythrocyte-derived magnetic immune particle with CR1 and/or GYPA inactivation.

In addition, human plasma samples were cultured with various pathogenic substances (MRSA, ESBL(+) E. coli, RSV, CMV, ZIKV E Protein, HCoV-OC43 (Human coronavirus OC43), or SARS-COV-2 S Protein), respectively, and inoculated to a concentration of about 10⁴ CFU/mL for bacteria and about 104 PFU/mL for viruses, protein was inoculated to a concentration of about 1 μg/mL), and after injecting each of the various types of magnetic immune particle obtained above, the pathogenic substance removal rate (%) of each magnetic immune particle was measured. The specific experimental method is the same as the method performed in Experimental Example 1, and the type of magnetic immune particle injected are as follows:
1) Erythrocyte-derived magnetic immune particle (RBC-MNVs) prepared in Example 1 above.
2) Magnetic immune particle in which the cell membrane surface molecule CR1 is inactivated (CR1 blocked RBC-MNVs) in the erythrocyte-derived magnetic immune particle prepared in Example 1 above.
3) Magnetic immune particle in which the cell membrane surface molecule GYPA is inactivated (GYPA blocked RBC-MNVs) in the erythrocyte-derived magnetic immune particle prepared in Example 1 above.
4) Magnetic immune particle in which the cell membrane surface molecules CR1 and GYPA are inactivated (GYPA&CR1 blocked RBC-MNVs) in the erythrocyte-derived magnetic immune particle prepared in Example 1 above.
5) Magnetic particle (MNPs) that do not include a cell membrane surface.

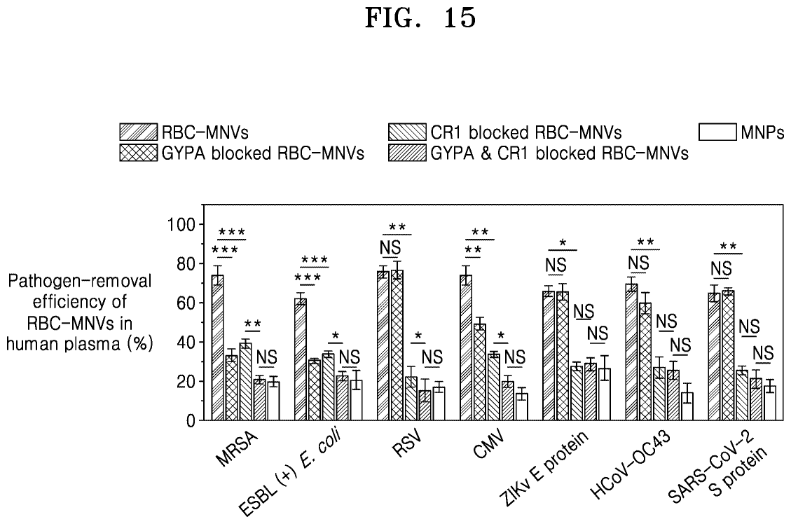

As a result, as shown in FIG. 15, it was confirmed that when the cell membrane surface molecules CR1 or GYPA are inactivated in the erythrocyte-derived magnetic immune particle prepared in Example 1, the pathogenic substance removal rate by the erythrocyte-derived magnetic immune particle is reduced. Specifically, it was confirmed that inactivation of the cell membrane surface molecule CR1 significantly reduced the capture ability (or removal ability) of the erythrocyte-derived magnetic immune particle against MRSA, ESBL(+) E. coli, RSV, CMV, ZIKV E Protein, HCoV-OC43, and SARS-COV-2 S Protein, and it was confirmed that inactivation of the cell membrane surface molecule GYPA significantly reduced the capture ability (or removal ability) of the erythrocyte-derived magnetic immune particle against MRSA, ESBL(+) E. coli, and CMV. coli, and CMV, while the capture ability (or removal ability) of the erythrocyte-derived magnetic immune particle against RSV, ZIKV E Protein, HCoV-OC43, and SARS-COV-2 S Protein did not change significantly. In addition, it was confirmed that when both CR1 and GYPA, which are cell membrane surface molecules, are inactivated, the removal rate of pathogenic substances by the erythrocyte-derived magnetic immune particle was most reduced. In particular, it was found that in the erythrocyte-derived magnetic immune particle, CR1 and/or GYPA (particularly CR1) present on the cell membrane surface greatly contributes to capturing pathogenic substances.

Therefore, through the present experimental example, it was found that erythrocyte-derived magnetic immune particle prepared using erythrocyte-derived cell membranes in which CR1 and/or GYPA are expressed (or overexpressed) may significantly increase pathogenic substance capture ability (detection or removal ability).

### Experimental Example 7. Confirmation of Effect of Supplementing Opsonin to Improve Pathogenic Substance Capture Ability (Detection or Removal Ability) of Erythrocyte-Derived Magnetic Immune Particle

The effect of improving the pathogenic substance capture ability (detection or removal ability) of erythrocyte-derived magnetic immune particle by adding opsonin was confirmed.

Specifically, various pathogenic substances (MRSA, ESBL(+) E. coli, RSV, CMV, and E. coli) were inoculated to TBS buffer or human blood samples. Subsequently, various opsonins (MBL (mannose binding lectin), FCN-1 (Ficolin-1), FCN-2, FCN-3, CL-10 (collectin-10), CL-11, C3b, or C1q (complement component 1q)) were injected, respectively, and then injected the erythrocyte-derived magnetic immune particle prepared in Example 1 above, and the pathogenic substance capture rate (or removal rate, %) of the magnetic immune particle was measured. The specific experimental method is the same as the method performed in Experimental Example 1 above, except that the opsonin was injected into TBS buffer or human blood sample.

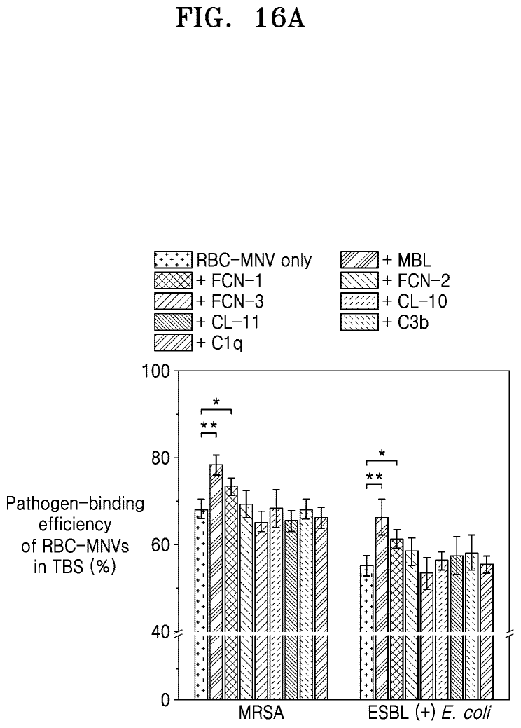

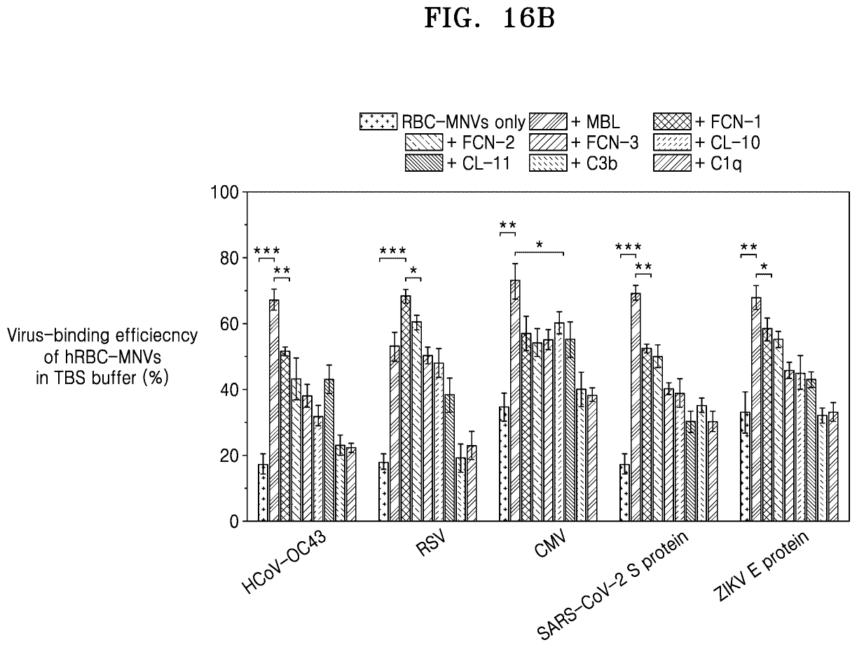

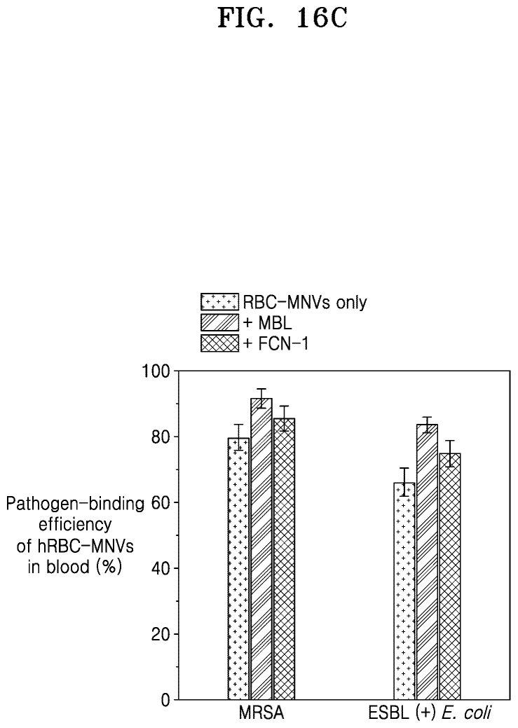

As a result, as shown in FIG. 16A, FIG. 16B, and FIG. 16C, it was confirmed that when the opsonins of MBL or FCN-1 were injected into a TBS buffer or human blood sample including pathogenic substances such as pathogenic bacteria, viruses, virus-derived antigenic proteins, and the like, together with the erythrocyte-derived magnetic immune particle prepared in Example 1 above, the pathogenic substance capture ability (detection or removal ability) of the erythrocyte-derived magnetic immune particle was significantly improved. In addition, it was confirmed that when the opsonins of FCN-2, FCN-3, CL-10, or CL-11 were injected into the TBS buffer including the pathogenic substance together with the erythrocyte-derived magnetic immune particle prepared in Example 1 above, the pathogenic substance capture ability (detection or removal ability) of the erythrocyte-derived magnetic immune particle was improved. However, when injecting C3b or C1q opsonin, it was confirmed that the level of increase in the pathogenic substance capture ability (detection or removal ability) of the erythrocyte-derived magnetic immune particle was relatively significantly low. Through this, it was found that specific opsonins, such as MBL or FCN-1, rather than all types of opsonins, may strongly enhance the pathogenic substance capture ability (detection or removal ability) of the erythrocyte-derived magnetic immune particle.

Through the present experimental example, it was found that in detecting or removing pathogenic substances (for example, pathogenic bacteria, virus, virus-derived antigenic protein, etc.) in a subject's sample (for example, blood, etc.) using erythrocyte-derived magnetic immune particle, when further injecting a specific opsonin such as MBL, FCN-1, FCN-2, FCN-3, CL-10, CL-11, etc. (especially, MBL and/or FCN-1), the pathogenic substance capture ability (detection or removal ability) of the erythrocyte-derived magnetic immune particle may be further improved, thereby further improving the detection or removal effect of pathogenic substance in the sample and the diagnosis or treatment effect of infectious diseases caused thereby.

### Experimental Example 8. Evaluation of Capture Ability (Detection or Removal Ability) of Erythrocyte-Derived Magnetic Immune Particle for Cancer-Related Substance

The applicability of erythrocyte-derived magnetic immune particle for the diagnosis, prevention, amelioration, or treatment of cancer was analyzed by determining whether the erythrocyte-derived magnetic immune particle may capture (detect or remove) cancer-related substances present in a blood sample in vitro.

Specifically, breast cancer cell line-derived extracellular vesicles, normal cell-derived nucleic acids, or tumor cell-derived nucleic acids were inoculated into human plasma or human blood samples and then mixed and reacted for a period of time. Then, each of the above samples was injected with the human erythrocyte-derived magnetic immune particle (or mouse erythrocyte-derived magnetic immune particle) prepared in Example 1 above, and after a reaction of about 20 minutes at about 37° C., the magnetic immune particle in each sample were fixed at a specific position using a magnet to prevent the magnetic immune particle from being included in the supernatant, and the supernatant was collected. The amount of breast cancer cell line-derived extracellular vesicles, normal cell-derived nucleic acids, or tumor cell-derived nucleic acids in the supernatant was measured, and the removal efficiency (in other words, detection rate or capture rate (binding efficiency)) of the magnetic immune particle to the breast cancer cell line-derived extracellular vesicles, normal cell-derived nucleic acids, and tumor cell-derived nucleic acids initially inoculated into each sample was determined by comparing the measured value with the amount of breast cancer cell line-derived extracellular vesicles, normal cell-derived nucleic acids, or tumor cell-derived nucleic acids.

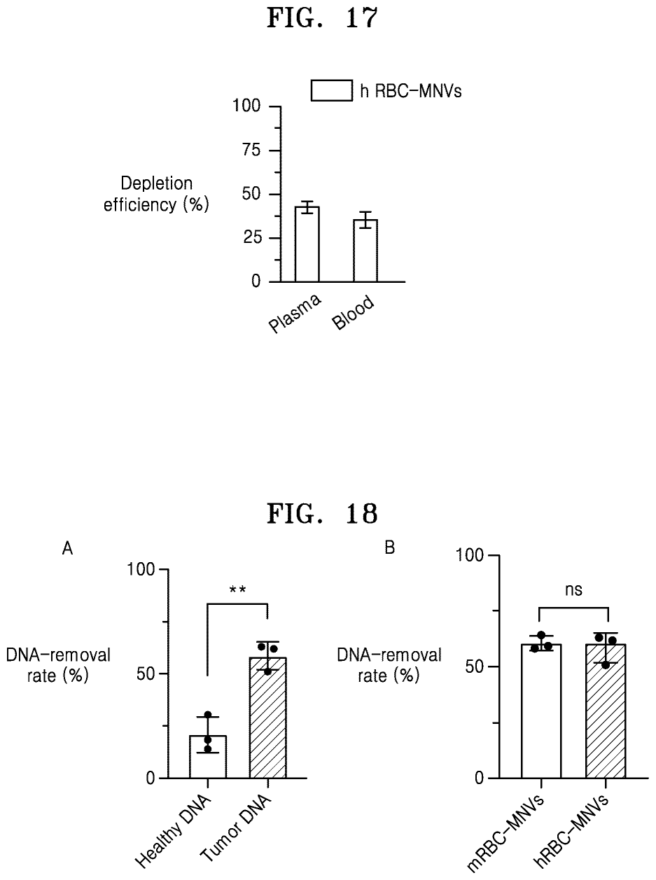

As a result, as shown in FIG. 17 and FIG. 18, it was confirmed that the erythrocyte-derived magnetic immune particle prepared in Example 1 above bind with excellent efficiency to cancer cell line-derived extracellular vesicles and tumor cell-derived nucleic acids present in human plasma or human blood samples, indicating that the erythrocyte-derived magnetic immune particle may detect or remove cancer-related substances such as cancer cell line-derived extracellular vesicles and tumor cell-derived nucleic acids present in human plasma or human blood with excellent efficiency. Furthermore, the erythrocyte-derived magnetic immune particle prepared in Example 1 had a low binding rate to normal cell-derived nucleic acids, but a significantly higher binding rate to tumor cell-derived nucleic acids, indicating that the erythrocyte-derived magnetic immune particle may selectively capture and remove only cancer-related substances such as cancer cell line-derived extracellular vesicle and tumor cell-derived nucleic acids present in human plasma or human blood, thereby enabling cancer-specific detection, diagnosis of cancer (for example, diagnosis of cancer metastasis or cancer recurrence, prediction of drug response to cancer therapeutics, etc.), or prevention or treatment of cancer (for example, prevention or treatment of cancer metastasis or cancer recurrence, improvement of drug response to cancer therapeutics, improvement of prognosis of cancer treatment, adjuvant treatment in combination with cancer therapy, etc.). Furthermore, it was confirmed that both human erythrocyte-derived magnetic immune particle and mouse erythrocyte-derived magnetic immune particle bind tumor cell-derived nucleic acids present in human blood samples with excellent efficiency, indicating that erythrocyte-derived magnetic immune particle may be applied without species differences in the detection or removal of cancer-related substances.

Next, commercially available elution buffer for normal cell-derived nucleic acids and tumor cell-derived nucleic acids bound (in other words, captured) to the erythrocyte-derived magnetic immune particle prepared in Example 1 were used to recover the nucleic acids from the erythrocyte-derived magnetic immune particle, and the recovery rate was measured. Furthermore, the absorbance at 260 nm and 280 nm wavelengths was measured for the recovered nucleic acid, and the ratio was used to analyze the purity of the recovered nucleic acid.

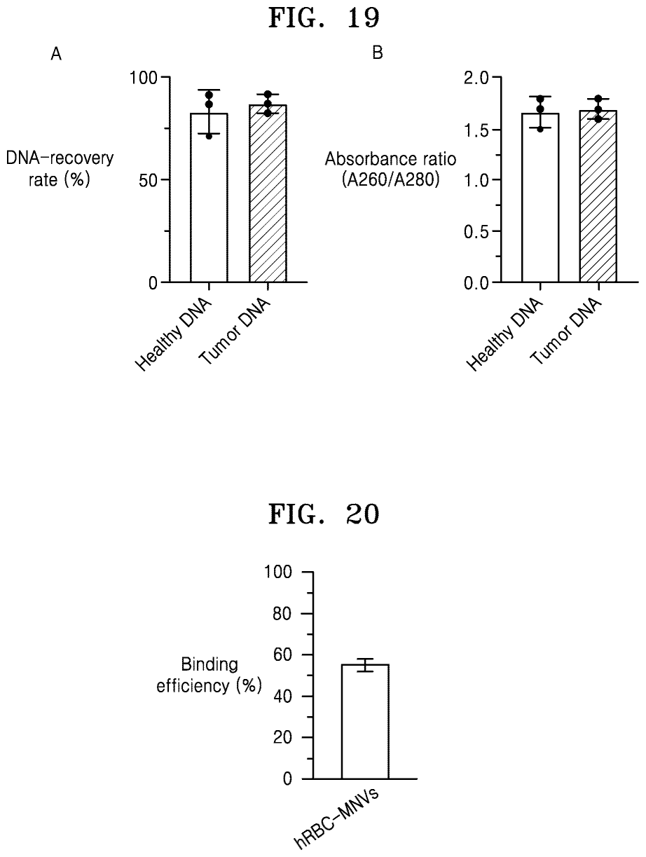

As a result, as shown in FIG. 19, it was confirmed that both normal cell-derived nucleic acids and tumor cell-derived nucleic acids bound (in other words, captured) to the erythrocyte-derived magnetic immune particle prepared in Example 1 were recovered by the above elution solution with an excellent efficiency of about 80% or more, and in addition, it was confirmed that the above absorbance ratio values of the recovered nucleic acids were about 1.677 (normal cell-derived nucleic acids) and about 1.7 (tumor cell-derived nucleic acids), confirming high DNA purity (generally, the above absorbance ratio value of pure DNA is between 1.6 and 1.8). Through this, it was found that when the erythrocyte-derived magnetic immune particle are used to detect cancer-related nucleic acids such as tumor cell-derived nucleic acids, the detected nucleic acids may be easily recovered with high purity, and molecular-level analysis such as PCR may be performed on the detected nucleic acids, thereby further improving the analysis efficiency and accuracy when the erythrocyte-derived magnetic immune particle are applied to cancer-specific detection and diagnosis of cancer, and the like.

### Experimental Example 9. Evaluation of Capture Ability (Detection or Removal Ability) of Erythrocyte-Derived Magnetic Immune Particle for Brain Disease-Related Substance

The applicability of erythrocyte-derived magnetic immune particle for the diagnosis, prevention, amelioration, or treatment of brain diseases (for example, Alzheimer's disease) was analyzed by determining whether the erythrocyte-derived magnetic immune particle may capture (detect or remove) brain disease-related substance present in a blood sample in vitro.

Specifically, amyloid beta 42 (Aβ42), an Alzheimer's-related protein, was inoculated into human plasma or human blood samples and then mixed and reacted for a period of time. Thereafter, the sample was injected with the human erythrocyte-derived magnetic immune particle prepared in Example 1 above, and after a reaction of about 20 minutes at about 37° C., the magnetic immune particle in the sample were fixed at a specific position using a magnet to ensure that the magnetic immune particles were not included in the supernatant, and the supernatant was collected. The amount of amyloid beta 42 in the supernatant was measured, and the removal efficiency (in other words, detection rate or capture rate (binding efficiency)) of the magnetic immune particle against amyloid beta 42 was measured by comparing the measured value to the amount of amyloid beta 42 inoculated into the initial sample.

In addition, the Alzheimer's-related proteins amyloid beta 42 (Aβ42), amyloid beta 40 (Aβ40), or tau protein, were inoculated into a mouse blood sample and then mixed and reacted for a period of time, in the same method as in Experimental Example 2 above, the reacted blood sample and the mouse erythrocyte-derived magnetic immune particle prepared in the same method as described in Example 1 were injected into the extracorporeal circulation device of Experimental Example 2 to remove the Alzheimer's-related proteins from the blood sample through erythrocyte-derived magnetic immune particle-based extracorporeal circulation. Afterwards, the concentration of the Alzheimer's-related protein in blood samples discharged at regular time intervals was measured.

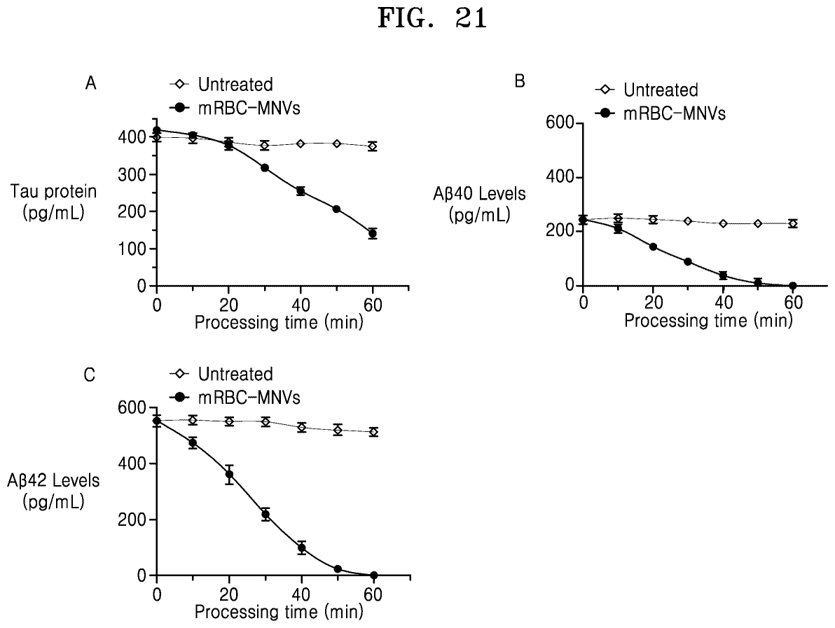

As a result, as shown in FIG. 20 and FIG. 21, it was confirmed that both the erythrocyte-derived magnetic immune particle prepared in Example 1 above and the mouse erythrocyte-derived magnetic immune particle prepared by the same method were able to remove amyloid beta 42, amyloid beta 40, amyloid beta 42, amyloid beta 40, and tau proteins present in the blood samples, indicating that the erythrocyte-derived magnetic immune particle may detect or remove brain disease (for example, Alzheimer's) related substances such as amyloid beta 42, amyloid beta 40, and tau proteins present in the blood with excellent efficiency. In particular, it was confirmed that using the above erythrocyte-derived magnetic immune particle-based extracorporeal circulation system, the tau protein present in the blood sample may be removed up to about 70% of the initial dose in about 1 hour, and amyloid beta 40 and amyloid beta 42 present in the blood sample may be removed in an amount close to about 100% of the initial dose in about 1 hour, which is below the detection limit of a commercially available enzyme-linked immunosorbent assay kit.

Through the present experimental example, it was found that the erythrocyte-derived magnetic immune particle may detect or remove brain disease (for example, Alzheimer's) related substances, such as amyloid beta 42, amyloid beta 40, and tau protein, present in the blood with excellent efficiency, and thus may be useful in the diagnosis, prevention, or treatment, etc. of brain disease (for example, Alzheimer's).

### Experimental Example 10. Comparison of Monodispersed Magnetic Immune Particle and Polydispersed Magnetic Immune Particle

The capture abilities (detection ability or removal ability) of monodispersed magnetic immune particle and polydispersed magnetic immune particle for cancer-related substance and brain disease-related substance present in in vitro blood samples were compared and analyzed.

First, monodisperse magnetic particle(s) were prepared and provided by BElement Inc. (South Korea). Additionally, polydisperse magnetic particle(s) were purchased and prepared (Cat: 02121, Ademtech, France). For the monodisperse magnetic particle(s) and polydisperse magnetic particle(s), the particle size (diameter) distribution and polydispersity index (PDI) were measured using Zeta size equipment (Nano ZS Zetasizer, Malvern analytical, UK). The polydispersity index is defined as the square of the standard deviation of the particle size (diameter) divided by the average of the particle size (diameter), and may have a value between 0 and 1, with a value closer to 0 (for example, about 0.17 or less) indicating that the particles are monodisperse particle with a high degree of uniformity in size.

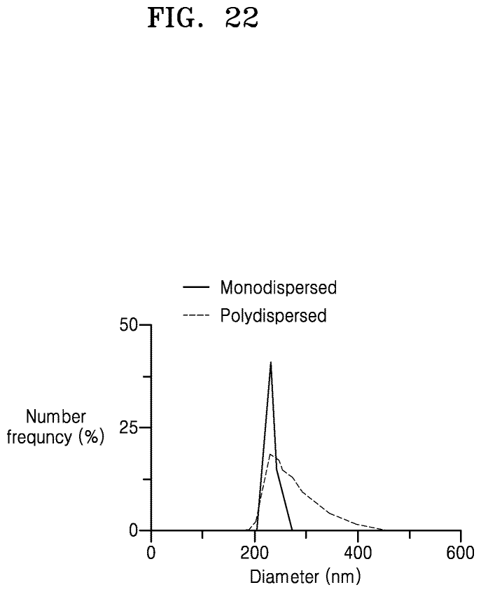

As a result, as shown in FIG. 22, it was confirmed that the monodisperse magnetic particle(s) has a diameter distribution measured between about 200 and 300 nm, and in particular, the polydispersity index is significantly lower than about 0.05 (specifically, about 0.03 or less, about 0.02 or less, or about 0.02), and the particle is highly uniform in size. On the other hand, it was confirmed for the polydisperse magnetic particle(s), the diameter distribution was measured to be between about 150 nm and about 500 nm, and the polydispersity index was also measured to be about 0.176, and the size of the particle(s) is highly non-uniform compared to the monodisperse magnetic particle(s).

Next, using the monodisperse magnetic particle(s) or polydisperse magnetic particle(s) and the erythrocyte-derived cell membrane, the erythrocyte-derived magnetic immune particle was prepared by the same method as described in Example 1 above. The magnetic immune particle prepared using the monodisperse magnetic particle(s) and erythrocyte-derived cell membrane was classified as a “monodisperse magnetic immune particle”, and the magnetic immune particle prepared using the polydisperse magnetic particle(s) and erythrocyte-derived cell membrane was classified as a “polydisperse magnetic immune particle”.

In addition breast cancer cell line-derived extracellular vesicle, normal cell-derived nucleic acid, circulating tumor cell-derived nucleic acid, amyloid beta 42, amyloid beta 40, or tau protein were inoculated into human blood samples, and then mixed and reacted for a period of time. Thereafter, each of the monodisperse magnetic immune particle and polydisperse magnetic immune particle were injected into each of the samples, and after a reaction of about 20 minutes at about 37° C., the magnetic immune particle in each sample was fixed at a specific position using a magnet to ensure that the magnetic immune particles were not included in the supernatant, and the supernatant was collected. The amount of the breast cancer cell line-derived extracellular vesicle, normal cell-derived nucleic acid, circulating tumor cell-derived nucleic acid, amyloid beta 42, amyloid beta 40, or tau protein in the supernatant was measured, comparing those measurements to the amount initially inoculated into each sample, the removal efficiency (in other words, detection rate or capture rate (binding efficiency)) of the magnetic immune particle to the breast cancer cell line-derived extracellular vesicle, normal cell-derived nucleic acid, circulating tumor cell-derived nucleic acid, amyloid beta 42, amyloid beta 40, or tau protein was measured.

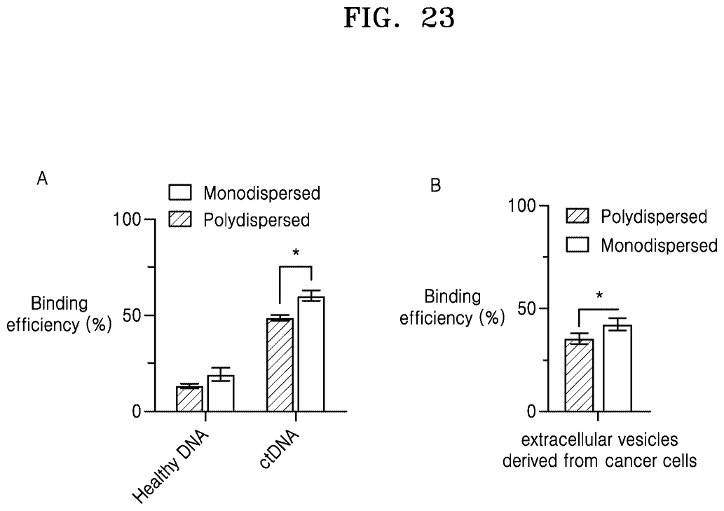

As a result, as shown in FIG. 23, it was confirmed that the monodisperse magnetic immune particle bind circulating tumor cell-derived nucleic acid and cancer cell line-derived extracellular vesicles present in human blood samples with excellent efficiency, and the binding rate to circulating tumor cell-derived nucleic acid was significantly higher than the binding rate to normal cell-derived nucleic acid. Through this, it was found that the monodisperse magnetic immune particle may specifically capture cancer-related substances present in human blood and detect or remove them with high efficiency. In particular, it was confirmed that the monodisperse magnetic immune particle exhibit significantly better capture ability (detection ability or removal ability) for cancer-related substance compared to the polydisperse magnetic immune particle.

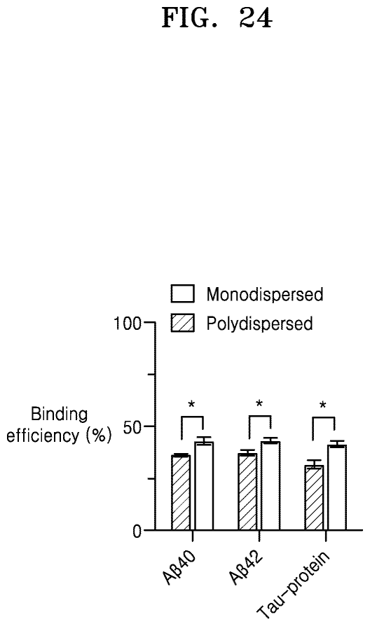

Furthermore, as shown in FIG. 24, it was confirmed that the monodisperse magnetic immune particle bind to amyloid beta 42, amyloid beta 40, and tau proteins present in human blood samples with excellent efficiency. Through this, it was found that the monodisperse magnetic immune particle may capture brain disease (for example, Alzheimer's disease) related substance present in human blood and detect or remove them with high efficiency. In particular, it was confirmed that the monodisperse magnetic immune particle exhibited better capture ability (detection ability or removal ability) of brain disease (for example, Alzheimer's) related substance compared to the polydisperse magnetic immune particle.

Through the present experimental example, it was found that erythrocyte-derived magnetic immune particle prepared using monodisperse magnetic particle(s) with a higher degree of particle size uniformity exhibited better capture ability (detection ability or removal ability) of cancer-related substance and brain disease-related substance compared to erythrocyte-derived magnetic immune particle prepared using polydisperse magnetic particle(s).

The specific aspects of the present disclosure have been described in detail, and such specific descriptions are merely illustrative embodiments to those skilled in the art, and do not limit the scope of the present disclosure.

It will be apparent to those skilled in the art that these specific descriptions are merely exemplary and that the scope of the present disclosure is not limited thereby. Accordingly, the substantial scope of the present disclosure is defined by the appended claims and their equivalents.

1. A magnetic immune particle comprising:
a cell membrane derived from an erythrocyte; and
a magnetic particle attached to the cell membrane.

2. The magnetic particle of claim 1, wherein the magnetic immune particle comprises one or more magnetic element selected from the group consisting of iron (Fe), nickel (Ni), cobalt (Co), manganese (Mn), bismuth (Bi), zinc (Zn), strontium (Sr), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), ruthenium (Lu), copper (Cu), silver (Ag), gold (Au), cadmium (Cd), mercury (Hg), aluminum (Al), gallium (Ga), indium (In), thallium (TI), and calcium (Ca), barium (Ba), radium (Ra), platinum (Pt), and lead (Pd).

3. The magnetic immune particle of claim 1, wherein the magnetic immune particle comprises an outer surface comprising a cell membrane and an inner core comprising the magnetic particle.

4. The magnetic immune particle of claim 1, wherein the cell membrane has the form of a vesicle.

5. The magnetic immune particle of claim 1, wherein the cell membrane comprises one or more type selected from the group consisting of a complement receptor (CR), a cluster of differentiation (CD) molecule, a glycophorin, a duffy antigen receptor for chemokines (DARC), glucose transporter, and monocarboxylate transporter.

6. The magnetic immune particle of claim 1, wherein the magnetic immune particle is obtained by extruding or sonicating a mixture of an erythrocyte or a cell membrane isolated from the erythrocyte and the magnetic particle.

7. The magnetic immune particle of claim 6, wherein the magnetic particle is a monodisperse magnetic particle.

8. The magnetic immune particle of claim 7, wherein the monodisperse magnetic particle has a polydispersity index (PDI) of 0.17 or less.

9. The magnetic immune particle of claim 1, wherein the magnetic particle is a monodisperse magnetic particle.

10. The magnetic immune particle of claim 9, wherein the monodisperse magnetic particle has a polydispersity index (PDI) of 0.17 or less.

11. A composition comprising a magnetic immune particle of claim 1,
wherein the composition is for detecting or removing at least one type selected from the group consisting of a pathogenic substance, inflammatory cytokine, blood glucose, cancer-related substance, and brain disease-related substance.

12. The composition of claim 11, wherein the pathogenic substance comprises at least one type selected from the group consisting of bacteria, fungi, virus, parasite, prion, and toxin.

13. The composition of claim 11, wherein the inflammatory cytokine comprises at least one selected from the group consisting of tumor necrosis factor-α (TNF-α), interleukin 4 (IL-4), interleukin 6 (IL-6), interleukin-1 beta (IL-1β), interleukin-1 alpha (IL-1α), interleukin 8 (IL-8), interferon gamma (IFN-γ), and granulocyte-macrophage colony-stimulating factor (GM-CSF).

14. The composition of claim 11, wherein the cancer-related substance comprises a cancer cell, a cancer cell-derived extracellular vesicle, a cancer cell-derived nucleic acid, or a combination thereof.

15. The composition of claim 11, wherein the brain disease-related substance comprises an amyloid beta (Aβ) protein, a tau protein, or a combination thereof.

16. A composition for the diagnosis of a disease including the magnetic immune particle of claim 1,
wherein the disease is an infectious disease, an inflammatory disease, diabetes, cancer, or a brain disease.

17. A method of detecting or removing, from a sample, at least one type selected from the group consisting of a pathogenic substance, an inflammatory cytokine, blood glucose, a cancer-related substance, and a brain disease-related substance present in the sample,
the method, comprising: contacting and mixing the sample with a magnetic immune particle of claim 1; and applying a magnetic field to the mixed sample.

18. The method of claim 17, further comprising introducing opsonin prior to applying the magnetic field.

19. The method of claim 17, wherein the method is performed by an extracorporeal circulation device.

20. A method for providing information necessary for the diagnosis of a disease, comprising: contacting and mixing the magnetic immune particle of claim 1 with a sample isolated from a subject; and applying a magnetic field to the mixed sample,
wherein the disease is an infectious disease, an inflammatory disease, diabetes, cancer, or a brain disease.

21. The method of claim 20, wherein the method further comprises introducing opsonin prior to applying a magnetic field.

22. The method of claim 20, wherein the method is performed by an extracorporeal circulation device.

23. A method of preventing or treating a disease in a subject, comprising: contacting and mixing the magnetic immune particle of claim 1 with a sample isolated from the subject to provide a mixed sample; applying a magnetic field to the mixed sample to remove the magnetic immune particle from the mixed sample; and injecting the sample from which the magnetic immune particle has been removed back into the subject,
wherein the disease is an infectious disease, an inflammatory disease, diabetes, cancer, or a brain disease.

24. The method of claim 23, wherein the method further comprises introducing opsonin prior to applying the magnetic field.

25. The method of claim 23, wherein the method is performed by an extracorporeal circulation device.

26. A method of preparing a magnetic immune particle, comprising: mixing an erythrocyte or cell membrane isolated from the erythrocyte with a magnetic particle; and
extruding or sonicating a mixture obtained in the mixing.

27. The method of claim 26, wherein the magnetic particle is a monodisperse magnetic particle.

28. The method of claim 27, wherein the monodisperse magnetic particle has a polydispersity index of 0.17 or less.
